(19) United States
(12) Patent Application Publication
(10) Pub. No.: US 2010/0150991 A1
(43) Pub. Date: Jun. 17, 2010

(54) Combination Wound Therapy

(76) Inventor: Brent H. BERNSTEIN, Bethlehem, PA (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(21) Appl. No.: 12/622,068

(22) Filed: Nov. 19, 2009

Related U.S. Application Data
(60) Provisional application No. 61/122,457, filed on Dec. 15, 2008.

(51) Int. Cl. A61K 33/00; A61K 9/70; A61K 33/04; A61M 1/00

(52) U.S. Cl. 424/447; 424/700; 424/699; 424/613; 424/708; 424/718; 604/23

(57) ABSTRACT

A device for providing improved wound healing is described. The device includes a vacuum system for applying a sub-atmospheric pressure to the wound, a gas supply system for applying a gaseous wound healing agent to the wound, and a controller connected with the vacuum system and the gas supply system that controls the applications of the sub-atmospheric pressure and the application of the gaseous wound healing agent to the wound. A method of using the device for improved wound healing is also described.

### CROSS-REFERENCE TO RELATED APPLICATIONS

This application is entitled to and claims the benefit of the priority pursuant to 35 U.S.C. §119(e) of U.S. Provisional Patent Application No. 61/122,457, filed Dec. 15, 2008, the disclosure of which is hereby incorporated by reference in its entirety.

### BACKGROUND OF THE INVENTION

The present invention relates generally to wound treatment involving a combined therapy of negative pressure wound treatment and medical gas insufflations.

Non-healing wounds are a problem. A chronic wound is defined as one that fails to process through the orderly phases of wound healing in a timely fashion. The first stage of wound healing is the hemostasis phase where clot is formed to limit blood loss and provisional matrix is laid down for later cellular infiltration. Initial vasoconstriction occurs secondary to catacholimines released during this phase. Platelet aggregation and adherence occurs, and they release cytokines, growth factors, ADP, and ATP. Fibrin formation stabilizes the clot and interacts with fibronectin to allow for cell migration. Chronic wounds are many times “stuck” in one of the following mid-stages. Stalled wound healing is an identified problem.

Even when a wound progresses normally through the phases of wound healing, certain problems can exist that need to be addressed. One such problem is the potential for contamination of the wound with debris or bacteria. Another problem is the pain associated with multiple daily dressing changes. Excess drainage from the wound can cause problems in hygiene and breakdown of intact peri-wound skin. Finally, the tendency of the wound edges to retract and allow enlargement of the wound is an identified problem.

The inflammatory phase is a protective tissue response to dilute, wall off, and destroy outside agents. The inflammatory phase is also known as the lag phase. There is initial vasodilation for 3-4 days caused by release of prostaglandins, leukotrienes, and histamine by mast cells. Capillary leakage between gaps in the endothelial cells leads to edema. There is an influx of polymorphonuclear (PMN) cells which release enzymes and cytokines and phagocytize bacteria and debris. The delay in recruitment of the different leukocytes (lymphocytes, monocytes, and neutrophils) leads to the alternate name for this phase (lag phase). Monocytes transform into macrophages when they enter the extracellular matrix. They also phagocytize debris and bacteria and release enzymes and growth factors in large quantities. Both the macrophages and PMNs release nitric oxide which has antibacterial properties and also influence gene expression and cell differentiation. Reactive oxygen species are formed during the enzymatic processes and imbalances can cause abnormal healing. The hypoxic state noted centrally in most normal wounds induces a pro-angiogenic response that is initiated by the tissue macrophages. The rate of re-epithelization and collagen production is stimulated by and facilitated by a relatively hypoxic wound environment. The wound is dependent upon oxygen gradients for growth much like bone is dependent upon electrical gradients. The formation of a collagen matrix in granulation tissue is at optimal productivity at a low-partial pressure of oxygen (5-10 mmHg). Angiogenesis (capillary budding) is activated and fibroblasts begin to establish irregular patterns of collagen. Lack of an adequate hypoxic state in the central wound to create an oxygen gradient for wound healing is an identified problem. Lack of peri-wound vasodilation is another identified problem that can slow wound healing.

Bacteria induce a systemic effect on host wound healing. Cicatrix formation is higher in infected wounds and this was demonstrated by Carrell as well. Bacteria are necessary for the formation of granulation tissue. Tissues that are free of infection have a more orderly fibroblast arrangement and little if any scar formation. Bacteria and their byproducts can cause problems in the orderly sequence of wound healing and create a chronic wound. With the continuous inflammatory stimulus of these outside agents, a down regulation of the host's immune response can occur. They help create a hostile wound bed milieu with increased matrix metalloproteinases (MMPs) and inflammatory cytokines. This hostile environment leads to senescent (stalled) fibroblasts that show limited tendency towards mitosis which ultimately leads to suppression of fibroplasia. Overabundance of bacteria in the wound bed is an identified problem as are subsequent increased MMPs and inflammatory cytokines and repressed fibroplasia (mitosis). Generally, anything that stalls the wound in the inflammatory phase will prevent proliferation of tissue, increase exudate, and cause cells in the wound to become senescent.

The proliferative phase overlaps the tail end of the previous stage as it also begins on days 3-4 and continues into day 14-21. Fibroplasia reinforces injured tissue, neovascularization/angiogenesis establishes blood supply, and re-epithelialization covers the wound. Stabilization of newly formed microvasculature to prevent abnormal morphogenesis (and deficient microperfusion despite angiogenesis) and this is partly due to platelet derived growth factor. Inadequate angiogenesis and tissue perfusion are identified problems in wound healing. Epithelialization requires an appropriate provisional matrix and active keratinocytes that can respond to the biochemical signal. They begin to proliferate from the wound edge (or skin appendages) at 24 hours in normal wounds. The cells migrate via the process of desmosomes, hemidesmosomes, and basement membrane dissolution caused by matrix metalloproteinases.

Finally, the remodeling stage occurs via the balancing of collagen synthesis and breakdown of matrix which continues from three weeks to one year. The MMPs assist in this as well. Wounded tissue is replaced with connective tissue (scar) at the expense of structural strength. The resultant scar tissue has less tensile strength and energy absorption. At 14 days, tensile strength of skin approaches 35%.

Limitation of the inflammatory phase will minimize scar formation and may emulate tissue regeneration. Tissue regeneration is the process when structurally identical tissue replaces lost tissue. Epidermis, liver, and GI tract tissues retain this capability. Fetal wound healing is rapid and skips the inflammatory phase. The fetus is in an environment that is sterile, rich in hyaluronic acid and growth factors, and hypoxic. There is a decreased quantity of collagen deposition in the wound site during the initial phases of healing. Fetal wound healing will hold lessons for those interested in the healing of chronic wounds. Overabundance of scar tissue (collagen deposition) is therefore an identified problem. Elongation of the inflammatory phase (versus true tissue regeneration) in the wound healing cascade is an identified problem.

Delayed epithelization in a dry wound bed is an identified problem. Re-epithelization of moist wounds occurs faster than that of wounds allowed to dry and scab. Moist wounds have decreased neutrophils and increased macrophages than dry wounds. Moist wounds also have earlier cell migration and cell adhesion in the inflammatory stage with subsequent earlier release of growth factors. In addition, moist wounds have increased numbers of monocytes and lymphocytes adhering to the endothelium which assists in quicker migration of mononuclear cells into the wound.

Good wound care involves debridement, off-loading, infection control and moist dressings. Sharp debridement has been used historically to “re-start” the wound healing cascade by initiating bleeding, hemostasis, and platelet release of growth factors to re-create an acute wound. Historically, bacteria in the wound bed have been eliminated by debridement, irrigation, topical antiseptic solutions, topical antimicrobial dressings, topical antibiotics, oral antibiotics, and intravenous antibiotics. Moist dressings have also been used conventionally for wound healing, which can maintain a moist wound and control small to moderate amounts of exudate. However, there are some inherent problems with the conventional wound healing methods, such as limited ability to absorb exudate, need for daily dressing changes, inability to increase perfusion or mitosis, inability to pull together the wound edges (macrostrain) and inability to decrease peri-wound edema.

“Negative pressure wound therapy (NPWT),” also called “reduced pressure therapy,” or “vacuum therapy” uses sub-atmospheric pressure to promote or assist wound healing, or to remove fluids from a wound site. Reported benefits for NPWT include, for example, migration of epithelial and subcutaneous tissues, improved blood flow, removal of bacteria from the wound site, and micro-deformation of tissue at the wound site, which together result in increased development of granulation tissue and faster healing times. Negative pressure wound therapy utilizes a porous wound filler through which negative pressure is applied. The wound area may be covered with a semi occlusive clear drape and connected via a tube to a canister that is attached to a suction pump. The negative pressure system gently pulls out stagnant fluids, such as wound drainage or stagnant fluid surrounding the wound, and collects the stagnant fluids, such as in a sealed canister.

Although the NPWT technology has progressed over the years, there are currently only a few commercially active companies that supply vacuum or negative pressure devices for open wounds. Kinetic Concepts Inc. (KCI) (San Antonio, Tex., U.S.) uses the V.A.C.® (Vacuum Assisted Closure) system based on the technology described by Morykwas and Argenta. Blue Sky (Carlsbad, Calif., U.S.) utilizes the Versatile 1™ Wound Vacuum System based on the Chariker-Jeter technique. Other companies with devices similar to the Versatile 1™ Wound Vacuum System now exist. These competitor companies have thus far marketed their devices to utilize a simple gauze interface rather than foam. Some examples of these are the Exsudex™ Wound Drainage System by Synergy Healthcare (Derby, United Kingdom), the Invia® Healing System and outpatient Liberty™ from Medela Healthcare (Baar, Switzerland), Venture™ by Talley Medical (Lansing, Mich., U.S.), and the WoundASSIST® TNP by AnjoHuntleigh (Roselle, Ill., U.S.). Some of these newer systems are not yet currently approved in the United States by the FDA.

While reduced pressure treatment has been shown to enhance wound healing through macrostrain and microstrain of tissues, NPWT has not progressed to the point the phases of wound healing can be manipulated so that actual exudate production can be limited, collagen deposition can be modulated, scar limited, and vasodilation increased through precise dosing of topical medication. Also, current NPWT platforms do not allow for complex cycling of intermittent variable level negative pressure.

Combination of NPWT with topical liquid treatments has been used for wound treatment. For example, KCI had developed the V.A.C.® Instill System, which has the additional ability to allow gravity feed of solutions into a second smaller diameter tubing set and second T.R.A.C.® pad to fill the sponge construct with solution. The electronic controls have been modified to allow intermittent negative pressure therapy and solution feeding of the sponge. Combination of NPWT with topical fluid instillation had shown initial promise as a way of combining the benefits of NPWT with the ability to limit the viscosity of wound drainage, apply pain medications, and suppress bacteria with antiseptics and antibacterial medications. However, this platform has limitations. Firstly, the platform is prone to overextension of the dressing with fluid and subsequent leakage and loss of seal. The correct application of the dual-hosed dressing is subsequently problematic and prone to failure. The platform is prone to other errors in applications such as the need to elevate the fluid bag to allow gravity instillation of the medication. All of these inherent weakness has severely limited the use of the device in the marketplace (and than only in the acute care setting in a small number of institutions). Another major problem encountered by KCI and the users of the device are the limited number of fluids approved for topical use by the FDA. For instance, almost all antibiotics are not approved for topical use as a fluid. Much of the use of the device has therefore been with off-label use of medications.

The use of topical medical gases to treat open wounds is a relatively new idea and is still in its infancy. It is considered a passive therapy while NPWT is considered an active therapy. Examples of topical medical gases include, carbon dioxide (CO₂), oxygen (O₂), nitric oxide (NO). The benefits of topical medical gas treatment are the ability to use signaling molecules to directly affect the underline mechanism of wound healing, such as vasodilation, inflammation, expression of matrix metalloproteinases, apoptosis, bacterial growth, collagen deposition, etc. However, the limited currently available technologies are passive delivery devices, that do not deliver the gas, allow proper diffusion of the gas over the wound, maintain a firm seal, manage exudate, protect the wound, and provide adequate mechanical stimulation of the wound.

There is a need in the art for wound healing technologies that allow physicians to have more active control and modulation of the wound healing cascade and in particular the inflammatory phase of wound healing.

### BRIEF SUMMARY OF THE INVENTION

It is now discovered that a combination of a negative pressure wound therapy with a topical application of a gaseous wound healing agent results in improved wound healing.

In one general aspect, the present invention relates to a device for promoting healing of a wound. The device comprises:
a. a wound filler adapted to be placed over the wound;
b. a fluid impermeable cover adapted to enclose the wound filler and the wound, wherein the periphery of the fluid impermeable cover is adapted to be sealed to tissue surrounding the wound;
c. a vacuum system adapted to apply a sub-atmospheric pressure to the wound, wherein the vacuum system is operably in communication with the wound filler;
d. a gas supply system adapted to apply a gaseous wound healing agent to the wound, wherein the gas supply system is operably in communication with the wound filler; and
e. a controller adapted for connection with the vacuum system and the gas supply system to control the application of the sub-atmospheric pressure and the application of the gaseous wound healing agent to the wound.

In another general aspect, the present invention relates to a method of promoting healing of a wound in a subject. The method comprises:

placing a wound filler over the wound;

enclosing the wound filler and the wound with a fluid impermeable cover, wherein the periphery of the fluid impermeable cover is sealed to tissue surrounding the wound;

applying a sub-atmospheric pressure to the wound from a vacuum system, wherein the vacuum system is operably in communication with the wound filler;

applying a gaseous wound healing agent to the wound from a gas supply system, wherein the gas supply system is operably in communication with the wound filler; and

controlling the applications of the sub-atmospheric pressure and the gaseous wound healing agent to the wound by a controller connected to the vacuum system and the gas supply system.

Other aspects, features and advantages of the invention will be apparent from the following disclosure, including the detailed description of the invention and its preferred embodiments and the appended claims.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 is a schematic diagram illustrating an embodiment of the present invention;

FIG. 2 is a perspective view of a device according to an embodiment of the present invention, showing the front view of the device with the top lid in the closed position;

FIG. 3 is a perspective view of a device according to an embodiment of the present invention, showing the front view of the device with the top lid in the open position;

FIG. 4 is a perspective view of a device according to an embodiment of the present invention, showing the right side view of the device with the top lid in the closed position;

FIG. 5 is a perspective view of a device according to an embodiment of the present invention, showing the left side view of the device with the top lid in the closed position;

FIG. 6 is a perspective view of a device according to an embodiment of the present invention, showing the top side view of the device with the top lid in the closed position;

FIG. 7 is a perspective view of a device according to an embodiment of the present invention, showing the bottom side view of the device;

FIG. 8 is a perspective view of a device according to an embodiment of the present invention, showing the back side view of the device with the top lid in the closed position;

FIG. 9 is a photograph of the KCI ActiV.A.C® in the prior art;

FIG. 10 is a photograph of the KCI Info V.A.C® in the prior art;

FIG. 11 is a photograph of the KCI Instill® in the prior art;

FIG. 12 is a photograph of the Smith and Nephew VISTA™ and EZCARE™ in the prior art;

FIG. 13a is a photograph of the Medela Invia® Vario in the prior art;

FIG. 13b is a photograph of the Medela Invia® Liberty in the prior art;

FIG. 14a is a photograph of the Exsudex™ in the prior art;

FIG. 14b is a photograph of the WoundAssist™ in the prior art;

FIG. 15 is a photograph of the Venturi® in the prior art;

FIG. 16 is a photograph of the Epiflo® in the prior art;

FIG. 17 is a photograph of the Topical Wound Oxygen Two 2™ in the prior art;

FIG. 18 is a photograph of the Carboflow® in the prior art;

FIG. 19 is a schematic diagram illustrating a device according to an embodiment of the present invention that has a gas conditioning unit;

FIG. 20 is a schematic diagram illustrating a device according to an embodiment of the present invention that has multiple sources of gas;

FIG. 21 is a schematic diagram illustrating a device according to an embodiment of the present invention that has a pulsation unit;

FIG. 22 is a schematic diagram illustrating a portable device according to an embodiment of the present invention;

FIG. 23 is a schematic diagram illustrating a device according to an embodiment of the present invention that has an odor filter;

FIG. 24 is a schematic diagram illustrating a device according to an embodiment of the present invention that has a single treatment line to a hub port;

FIG. 25 is a schematic diagram illustrating a device according to an embodiment of the present invention that has two treatment lines to two separate hub ports;

FIG. 26 is a schematic diagram illustrating a device according to an embodiment of the present invention that has two treatment lines conjoined with Y-connector to a single hub port;

FIG. 27 is a schematic diagram illustrating a device according to an embodiment of the present invention that has a single treatment line to a conduit port;

FIG. 28 is a schematic diagram illustrating a device according to an embodiment of the present invention that has two treatment lines to two separate conduit ports;

FIG. 29 is a schematic diagram illustrating a device according to an embodiment of the present invention that has two treatment lines with one to a hub port and one to a conduit port;

FIG. 30 is a schematic diagram illustrating a device according to an embodiment of the present invention that has a single treatment line split with a Y-splitter to both conduit and hub ports;

FIG. 31 is a schematic diagram illustrating a device according to an embodiment of the present invention that has two intelligent vacuum control algorithm sensors; and

FIG. 32 is a schematic diagram illustrating a device according to an embodiment of the present invention that has a bridge connector;

FIG. 33 is a schematic diagram illustrating a device according to an embodiment of the present invention that has a single treatment line to a conduit port in a sinus tract.

### DETAILED DESCRIPTION OF THE INVENTION

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

As used herein, the term “subject” refers to an animal, preferably a mammal, who/which has been the object of treatment, observation or experiment. Examples of a subject can be a human, a livestock animal (beef and dairy cattle, sheep, poultry, swine, etc.), or a companion animal (dog, cat, horse, etc).

The present invention relates to devices and methods for improved wound healing, which combines the negative pressure wound therapy with the topical use of a gaseous wound healing agent. Devices and methods according to embodiments of the present invention simultaneously meet many or all of the multiple goals selected from the following group: (1) protect the wound base from contamination and trauma; (2) maintain a moist, controlled wound; (3) allow application of intermittent negative and positive pressure to a wound; (4) allow application of intermittent negative pressure and ambient pressure to the wound; (5) allow application of intermittent variation of negative pressure levels to the wound; (6) remove bacteria or other microbes mechanically; (7) remove exudate mechanically; (8) provide macrostrain; (9) provide microstrain, increased fibroplasia, and increased mitosis; (10) increase local perfusion and angiogenesis via the mechanical properties of NPWT; (11) allow for diffusion of a gaseous topical material over the wound bed; (12) allow for combination gas diffusion to the wound bed and all of the benefits of NPWT; (13) allow for multiple different gas application to the wound either in combination, in cycles, or at different concentrations; (14) allow for combination of gas diffusion to the wound and NPWT in multiple different cycles of therapy including simultaneous therapy, positive gas diffusion, NPWT, and ambient pressure treatment; (15) allow for application of topical material to vasodilate wound and peri-wound vessels; (16) allow for application of topical material to limit the inflammatory phase of wound healing; (17) allow for application of topical material to limit or increase the deposition of collagen; (18) allow for application of topical material to limit or increase the activity of endogenous wound enzymes; (19) allow for application of topical material to affect apoptosis; (20) allow for application of topical material to affect the oxygen gradient of wounds; (21) allow for application of topical material to limit the production of exudate in the wound; (22) allow for application of topical material to suppress bacteria; (23) allow for application of topical material to without loss of dressing integrity; (24) allow for application of topical material that is precisely dosed; (25) allow acute care and outpatient therapy; (26) allow for limited number of dressing changes to prevent trauma, nursing hours, and pain; (27) allow for self-application by patient; (28) allow for epithelial migration while undergoing negative pressure wound therapy; (29) allow for utilization of either a foam packing material, gauze, or other wound filler to be with a soft, silicone drain tunneled under the fluid impermeable cover if desired; or hub port through the fluid impermeable cover; (30) reduce peri-wound edema; (31) allow for tissue regeneration; (32) allow for testing of the wound bed, exudate, and peri-wound tissue via sensors; (33) allow for the sensor data from the wound bed, exudate, and peri-wound tissue to control the cycles and pressures of wound therapy as well as the concentration and type of topical materials allowed to contact the wound bed; (34) allow for odor control at the wound interface; (35) allow for superior gas diffusion over the wound bed.

In a general aspect, a device according to an embodiment of the present invention comprises:
a. a wound filler adapted to be placed over the wound;
b. a fluid impermeable cover adapted to enclose the wound filler and the wound, wherein the periphery of the fluid impermeable cover is adapted to be sealed to tissue surrounding the wound;
c. a vacuum system adapted to apply a sub-atmospheric pressure (also known as negative pressure) to the wound, wherein the vacuum system is operably in communication with the wound filler;
d. a gas supply system adapted to apply a gaseous wound healing agent to the wound wherein the gas supply system is operably in communication with the wound filler; and
e. a controller adapted for connection with the vacuum system and the gas supply system to control the application of the sub-atmospheric pressure and the application of the gaseous wound healing agent to the wound.

The device according to an embodiment of the present invention generally includes a wound filler and a fluid impermeable cover that are used to fill and seal the wound, and a rigid housing containing a vacuum source to supply the sub-atmospheric pressure, a pressurized gas cylinder to apply the gaseous wound healing agent, and a controller that controls the operation of the pump and the gas cylinder. The wound filler is placed in direct communication with both the vacuum source for promotion of fluid drainage and other benefits of negative pressure wound therapy via suction, and the pressurized gas cylinder for topical insufflation of the wound with the gaseous wound healing agent at a slight positive flow. A drainage container is included in a device according to an embodiment of the present invention to allow collection of fluids removed from the wound by the vacuum source. The controller allows the user to control timing, duration, and cycles of the applications of either the negative pressure or the gaseous wound healing agent.

Devices according to embodiments of the present invention allow for maintenance of seal whether under negative or positive pressure cycles via series of valves and connectors. The device can have both a large unit version that accommodates medium to large size gas cylinder(s), which is suitable for the hospital use, as well as a smaller unit version that accommodates a smaller housing and gas cylinder(s), which is suitable for outpatient use.

The present device and method can be applied to chronic or acute wound. As used herein, the term “wound” refers to a trauma to any of the tissues of the body of a subject. The wound can be temporary or chronic. The wound can be endogenous, traumatic or iatrogenic in origin. It can be inflicted by any means, such as a disease or an injury that results in interruption or a breach of continuity of the skin and flesh of the subject. The wound can be caused by a surgical incision; a surgical wound dehiscence; an accident; a trauma; a pathological process, such as a metabolic disorder, an infection, or a vascular disorder; an assault, for example, by a weapon such as a gun or knife; bite wounds; post-amputation wounds, etc.

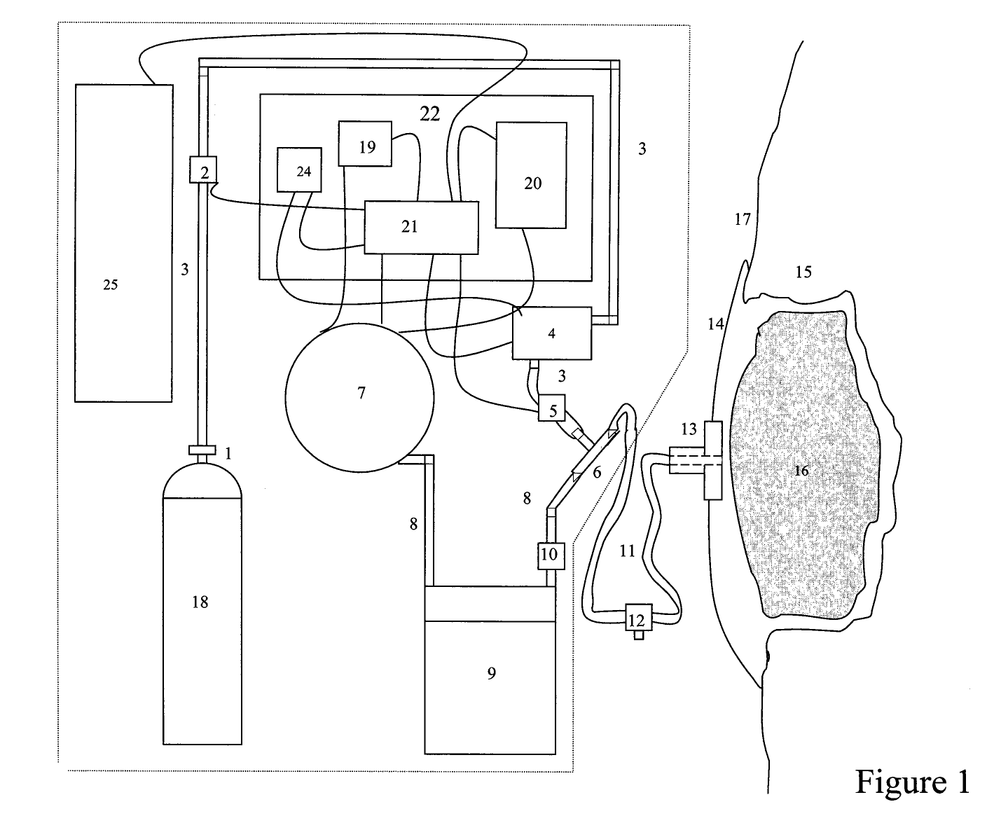

Referring to FIG. 1, there is shown a schematic view of a device according to an embodiment of the present invention for the treatment of a wound. The device allows topical insufflation of the wound with any gaseous wound healing agent at a slightly positive flow and concomitant administration of a negative pressure wound therapy. The device also allows intermittent application of both the vacuum suction and gas insufflation for stimulating wound healing in accordance with the present invention.

As shown in FIG. 1, a wound filler 16 is placed in an open wound site 15. It is readily understood by those skilled in the art that depending on the type of the wound to be treated, the wound filler can be applied over the wound in any manner known to those skilled in the art. For example, the wound filler can also be placed over a skin graft or flap at a wound site, etc.

The wound filler 16 can be any soft or rigid material of varying shape and size that is permeable to liquid and gas. It can be hydrophilic or hydrophobic. It can be disposable or reusable. The wound filler 16 provides compression to the wound 15, absorbs drainage, prevents motion, while allowing diffusion of the applied negative pressure and gaseous wound healing agents to the wound. The wound filler can be made of any suitable materials, such as cloth, gauzes (i.e., a thin, translucent fabric with a loose open weave), films, gels, foams, hydrocolloids, alginates, hydrogels and polysaccharide pastes, granules, keratin proteins, and beads. Any wound filler can also be treated with a substance, e.g., an anti-adherence agent, so as to prevent adherence of the filler to the wound bed. Any wound fillers that have been used or can be adapted for use in any NPWT can be used in the present invention. Exemplary wound fillers that can be used in the present invention include, but are not limited to, an open-cell foam pad such as polyurethane foam, a rigid porous screen, or a gauze. The wound filler is preferably sterilized and kept in sterile condition, e.g., wrapped in stile wrapping, prior to use.

In an embodiment of the present invention, the wound filler 16 is predisposed with one or more agents for promotion of increased wound healing. For example, the wound filler can be predisposed with one or more agents selected from the group consisting of a basic fibroblast growth factor or any other growth factor or cytokine that can enhance wound healing, and an anti-microbial substances. An anti-microbial substance reduces the possibility of infection, sepsis or putrefaction. It can be a germicide that kills or destroys a microbe, such as an antibiotic that kills bacteria, or a microbiostatic agent that only prevents or inhibits the growth of a microbe, such as a bacteriostatic that prevents the growth of bacteria.

Referring to FIG. 1, a fluid impermeable cover 14 is sealed circumferentially to peripheral intact skin 17 and covering the wound 15 and the wound filler 16. The fluid impermeable cover 14 isolates the wound 15 and the wound filler 16 from the environment. It creates a microenvironment where the negative pressure and the gaseous wound healing agent can be applied and retained, at least for a brief moment. The fluid impermeable cover 14 also protects the wound from external contamination, prevents drying and leaking, and retains the wound filler 16.

Any fluid impermeable covers that have been used or can be adapted for use in any NPWT can be used in the present invention. Exemplary fluid impermeable covers that can be used in the present invention, include, but are not limited to, a transparent dressing, whether being adhesive or nonadhesive. Additional control of the levels of the sub-atmospheric pressure and the gaseous wound healing agent applied to the wound site 15 can be achieved by adjusting the gas permeability of the fluid impermeable cover 14.

In a preferred embodiment of the present invention, the fluid impermeable cover 14 is substantially gas impermeable.

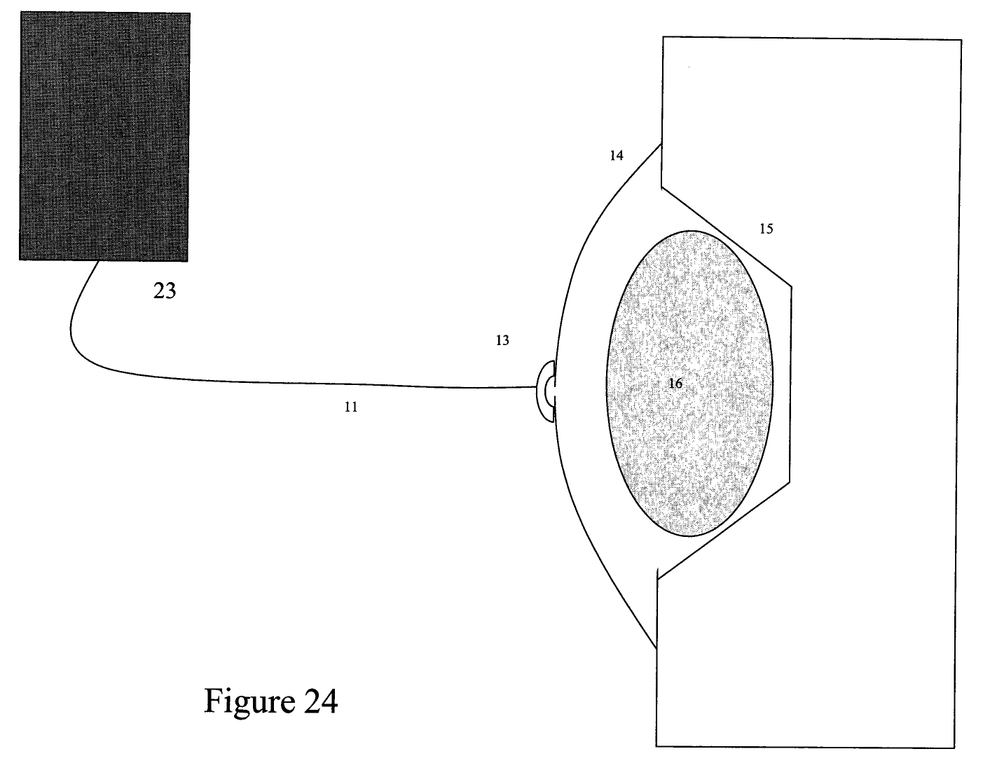

In an embodiment of the present invention, referring to FIG. 1 and FIG. 24, the fluid impermeable cover 14 is perforated. An adhesive-backed hub port 13, similar to the type of ports used in the KCI NPWT devices, is centered and sealed over the perforation. The hub port 13 is connected to a tubing 11, which continues into a rigid housing 23 and connects to a T-connector 6 for fluid and/or gas communication with the vacuum pump 7 and the pressurized gas cylinder 18. The conjoined tubing 11 and the hub port 13 place the wound filler 16 in liquid and/or gas communication with the vacuum pump 7 and/or the gas cylinder 18.

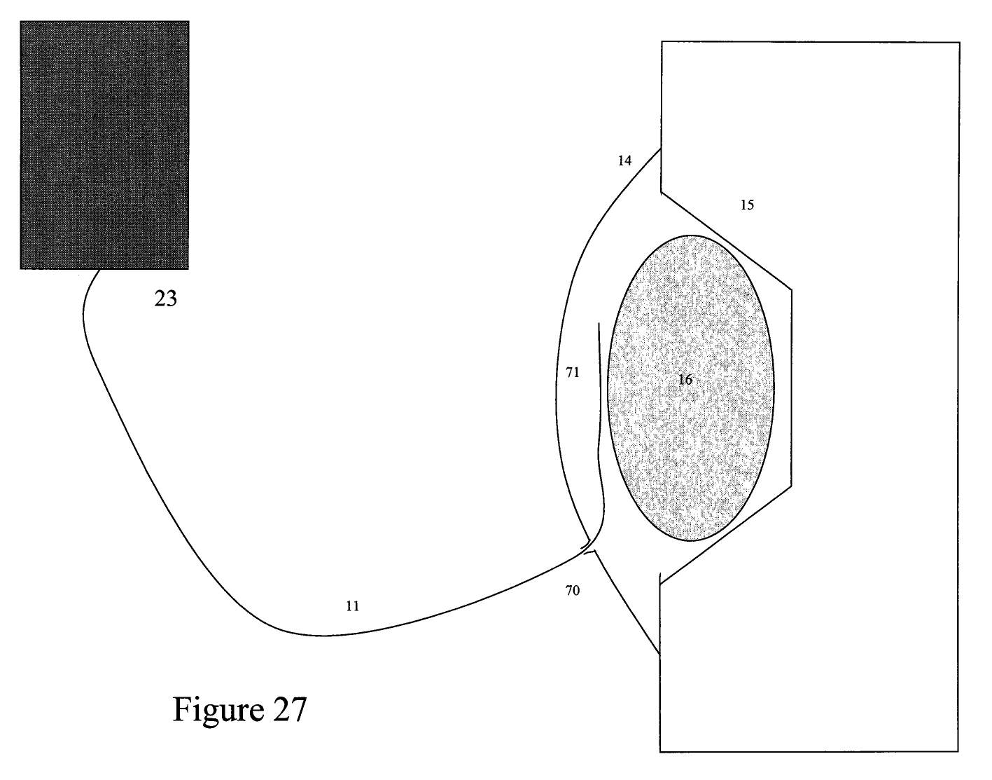

In another embodiment of the present invention, referring to FIG. 27, the conjoined tubing 11 of the vacuum system and the gas supply system connects to a soft conduit 71, which pierces through the fluid impermeable cover 14 and extends near, into, or under the filler 16. The conjoined tubing 11 and the conduit 71 are connected at a conduit port 70, which is similar to the type of ports used in the Chariker-type devices, in the fluid impermeable cover 14. The soft conduit 71 can be a soft silicone rubber, a plastic conduit, or any other soft conduits that can been used in NPWT. The soft conduit 71 is sealed to the fluid impermeable cover 14 with an adhesive drape. The soft conduit 71 places the wound filler 16 in liquid and/or gas communication with the vacuum pump and/or the gas cylinder.

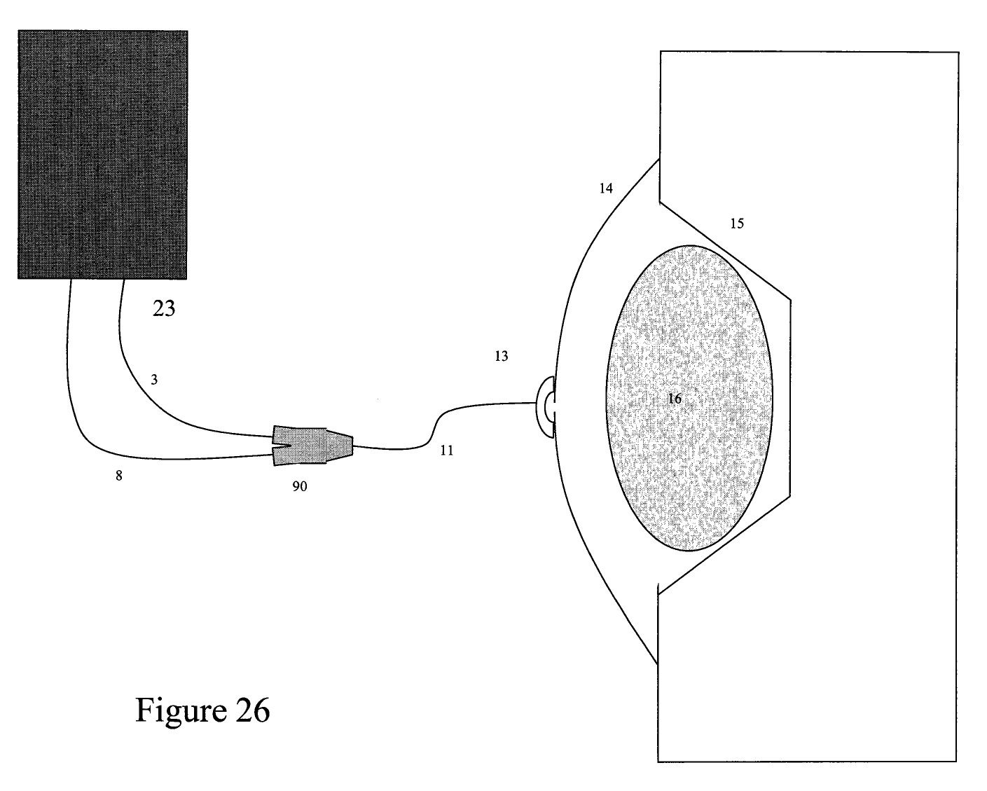

As illustrated in FIGS. 1, 24, 26 and 27, connecting the conjoined tubing 11 of the vacuum system and the gas supply system to a single port simplifies the operation. The vacuum system and the gas supply system can be conjoined with a T-connector either within or after exiting the rigid housing 23.

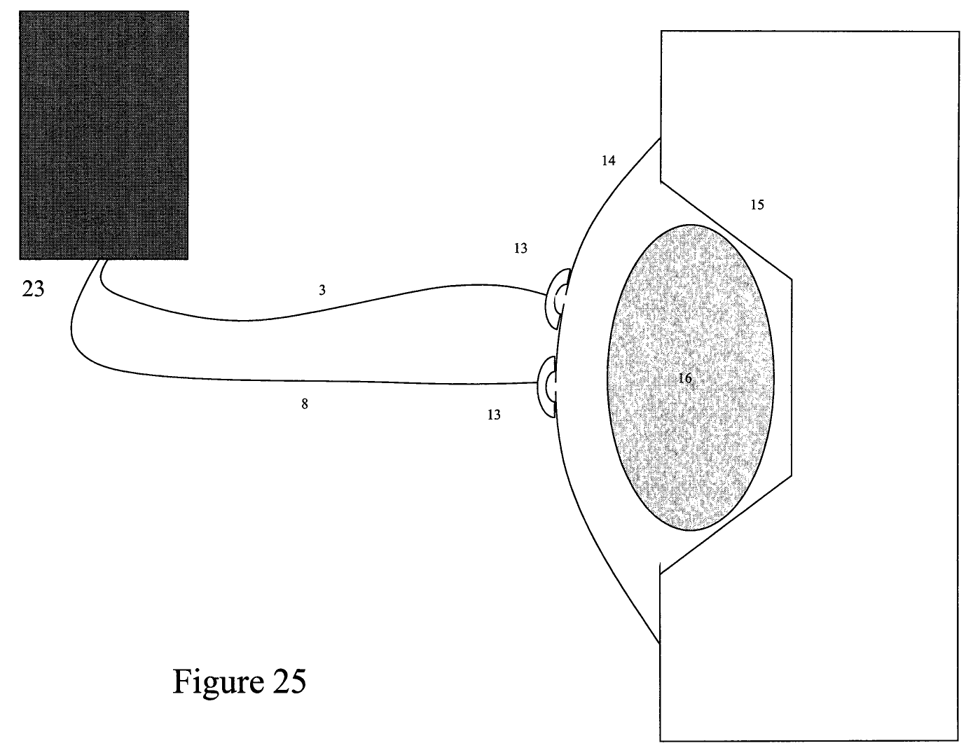

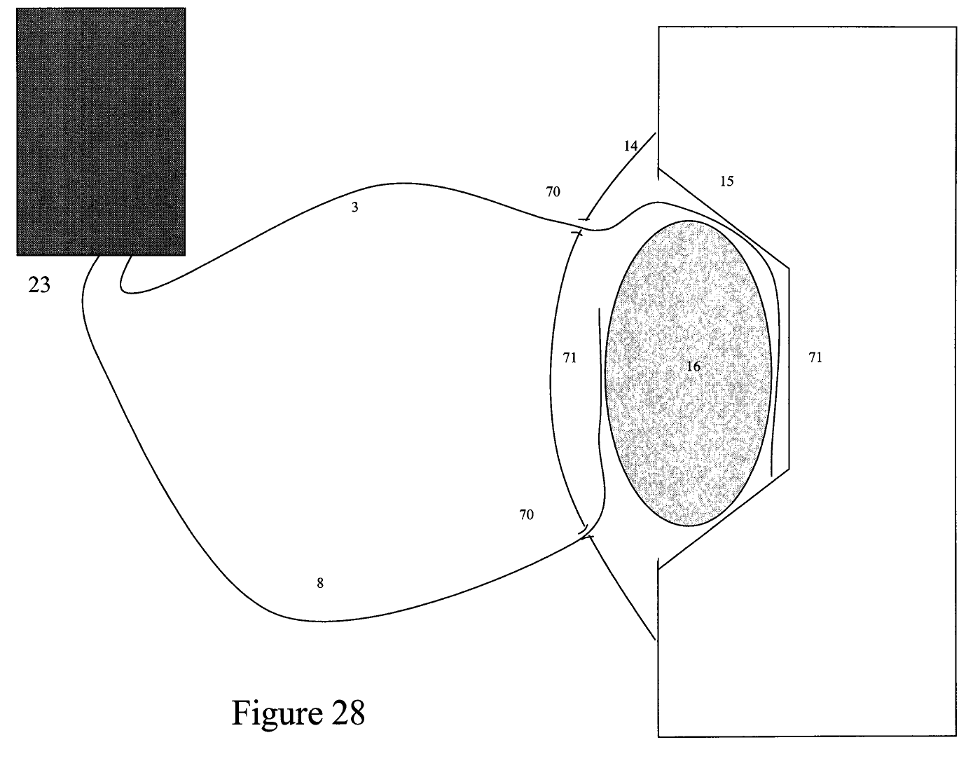

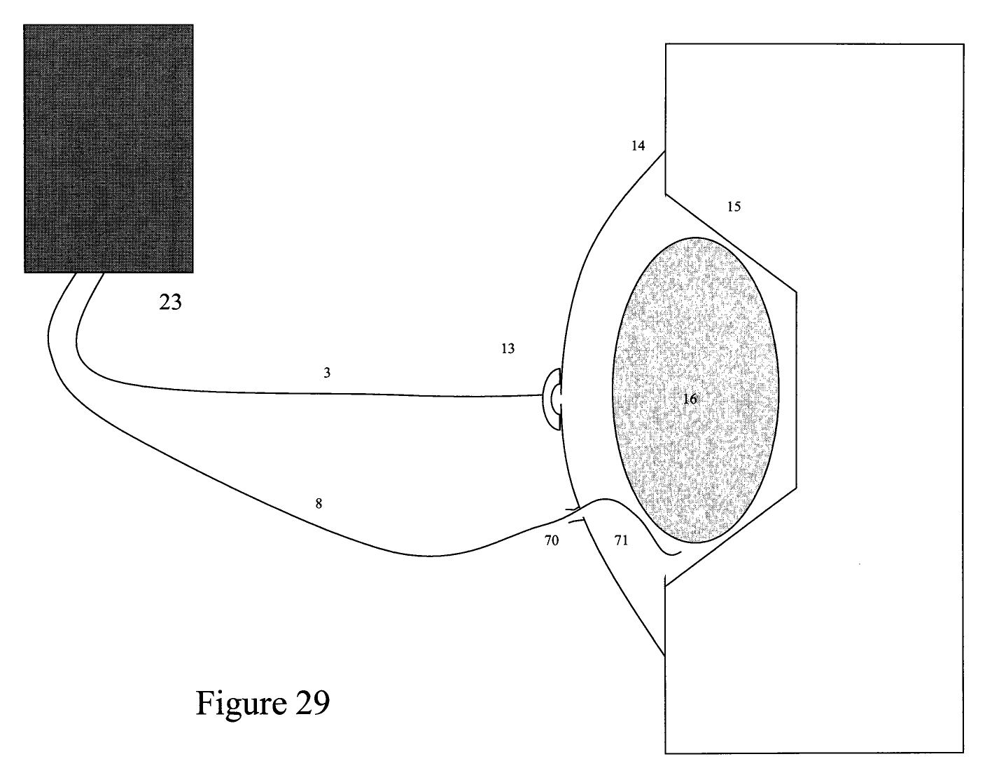

However, for additional flexibility, as illustrated in FIGS. 25, 28 and 29, the vacuum system and the gas supply system can also connect independently to the fluid impermeable cover 14 via separate ports in the fluid impermeable cover 14. The separate ports can be hub ports 13 (FIG. 25), conduit ports 70 (FIG. 28), or both hub port 13 and conduit port 70 (FIG. 29).

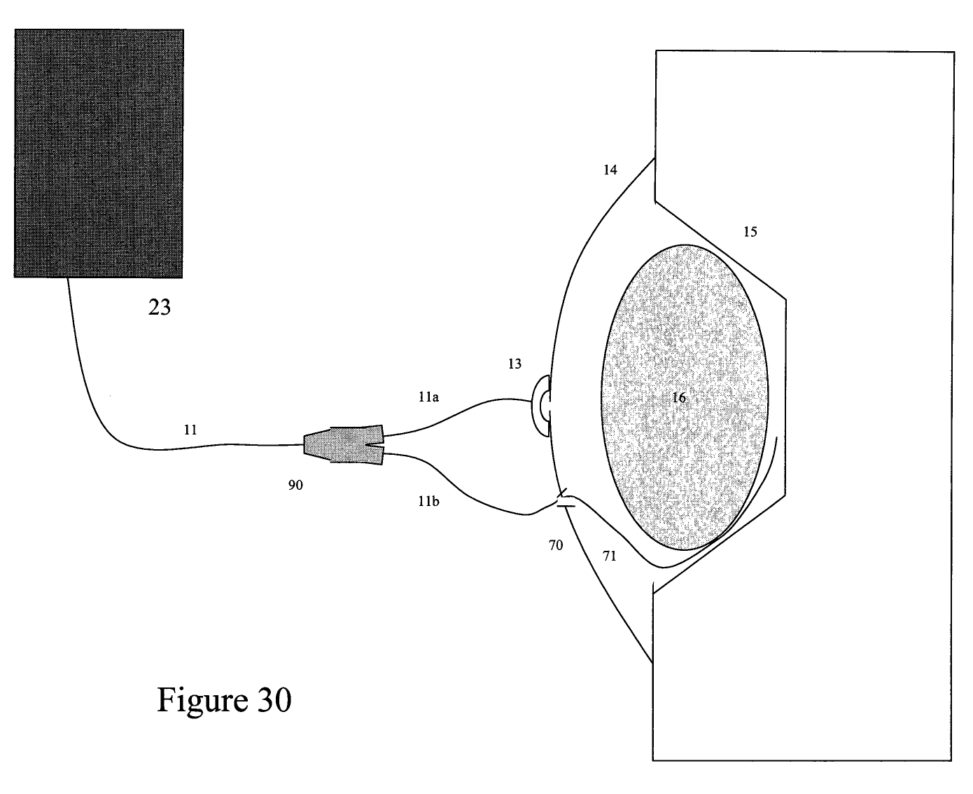

In another embodiment of the present invention, as illustrated in FIG. 30, the conjoined tubing 11 of the vacuum system and the gas supply system is split via a Y-splitter 90. The split tubes, 11a and 11b, connect to the fluid impermeable membrane 14 independently via separate ports, 13 and 70, respectively. The separate ports can be hub ports, conduit ports, or both. The conjoined tubing can be split either within or after exiting the rigid housing 23.

In another embodiment of the present invention, the vacuum system is operably in communication with the wound filler through multiple tubes that either are conjoined prior to connecting to the fluid impermeable membrane via a single hub port or a single conduit port, or connect independently to the fluid impermeable cover via separate ports.

In yet another embodiment of the present invention, the gas supply system is operably in communication with the wound filler through multiple tubes, such as those involving multi-gas supply units. The multiple tubes either are conjoined prior to connecting to the fluid impermeable membrane via a single hub port or a single conduit port, or connect independently to the fluid impermeable cover via separate ports. The separate ports can be hub ports, conduit ports, or both.

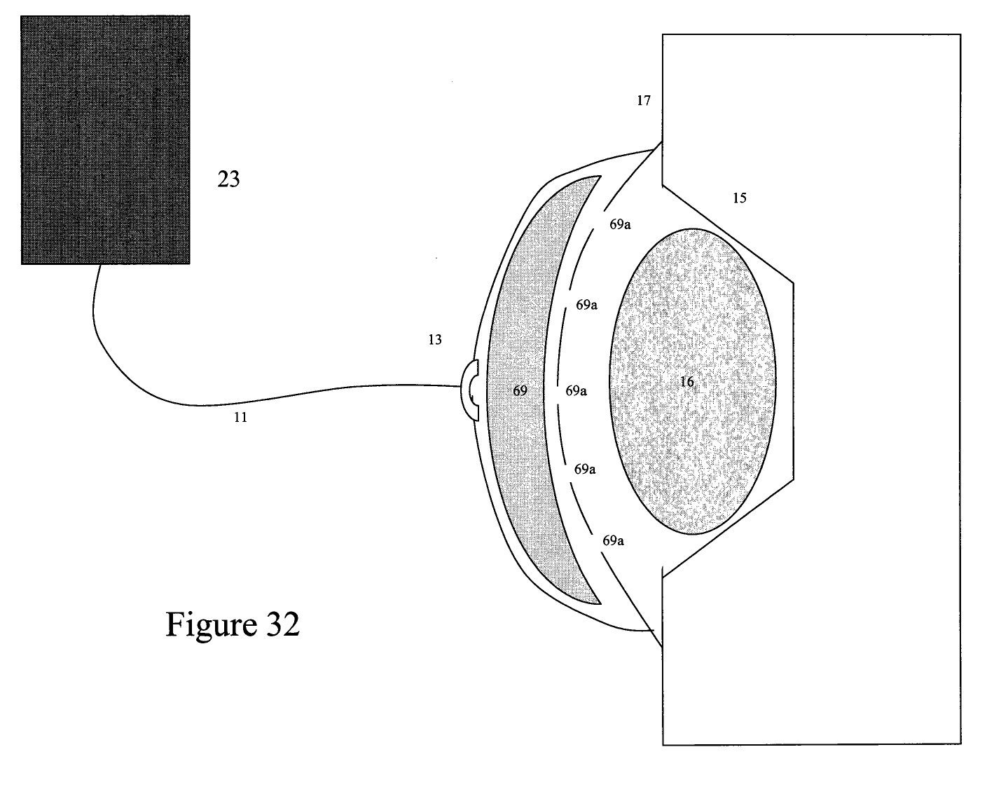

In still another embodiment of the present invention, referring to FIG. 32, the negative pressure and/or gaseous wound healing agent can be applied to the wound 15 through a bridge connector 69. The connector 69 acts as a “bridge” that readily conducts negative and/or positive pressure to be placed over the wound through multiple perforations 69a so as to allow more even distribution of negative or positive pressure over the large area. The use of multiple perforations is also preferred when the wound filler 16 packed within the wound cavity is less conductive to negative or positive pressure. The membrane connector can be connected to the conjoined vacuum system and the gas supply system or to the two systems independently.

In FIG. 1, a relief valve 12 is included within the conjoined tubing 11 to prevent over-pressurizing of the sealed wound filler/membrane construct and loss of seal and subsequent leakage. The miniature relief valve 12 can be incorporated in-line via either hose barb or female and male luer lock means.

Referring to FIG. 1, the device includes a vacuum system to apply a sub-atmospheric pressure to the wound. The vacuum system includes a vacuum pump 7 to generate negative pressure and to remove wound effluent, a container 9 to collect wound effluent removed by the vacuum pump 7, a connecting tubing 8 (also called negative pressure tubing) and a check valve 10.

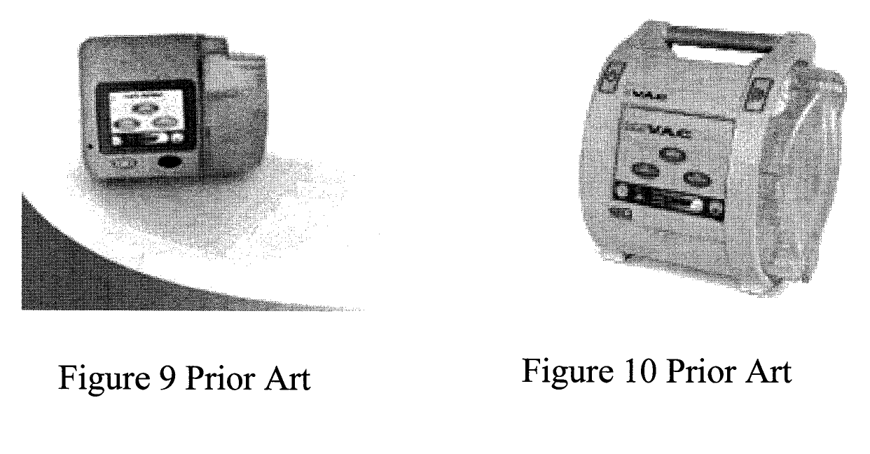

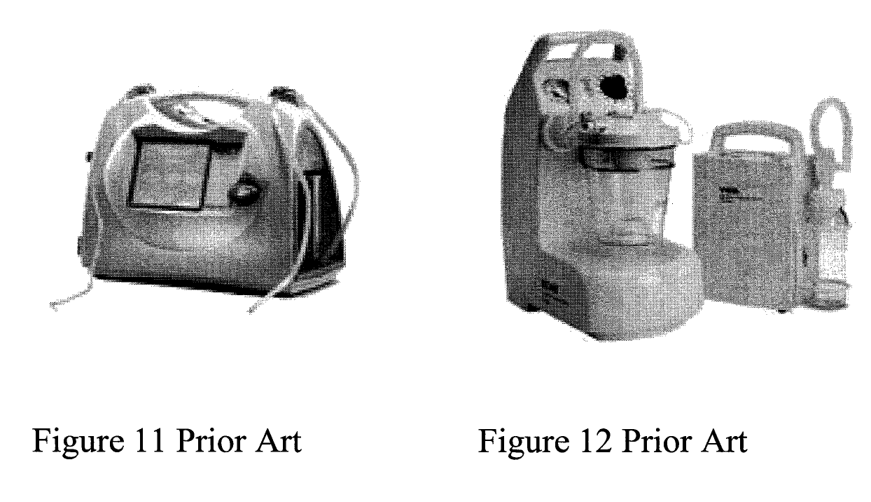

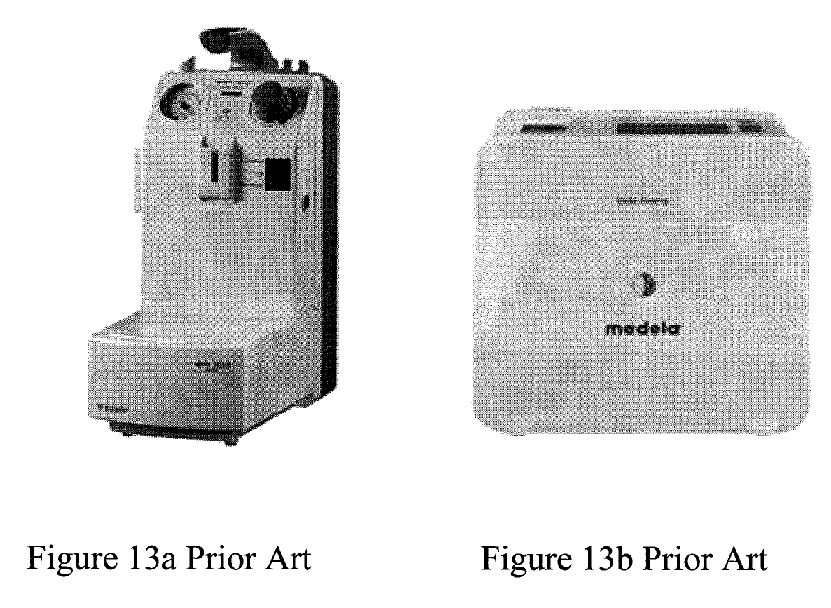

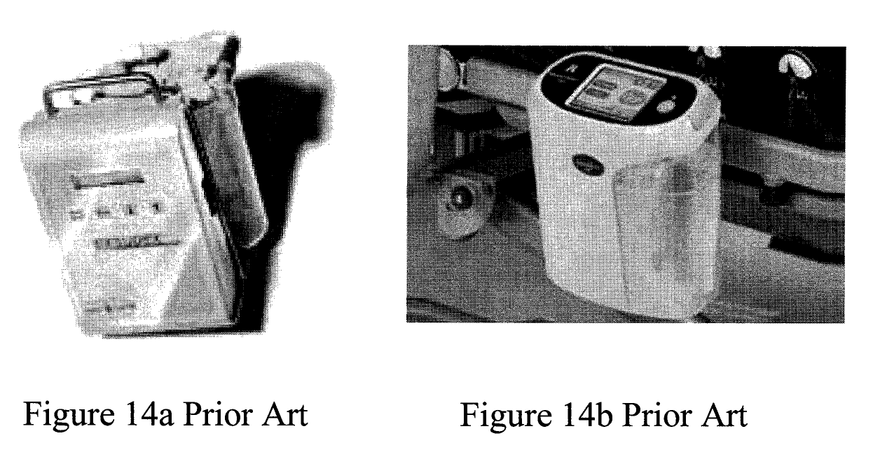

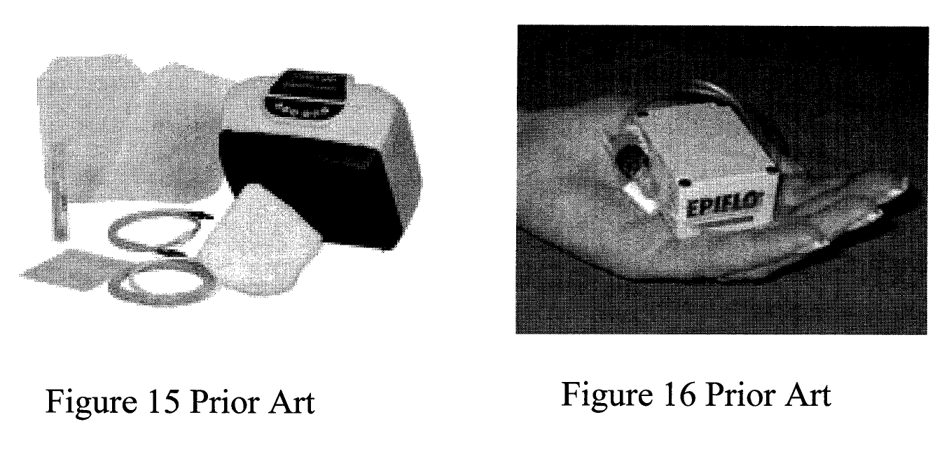

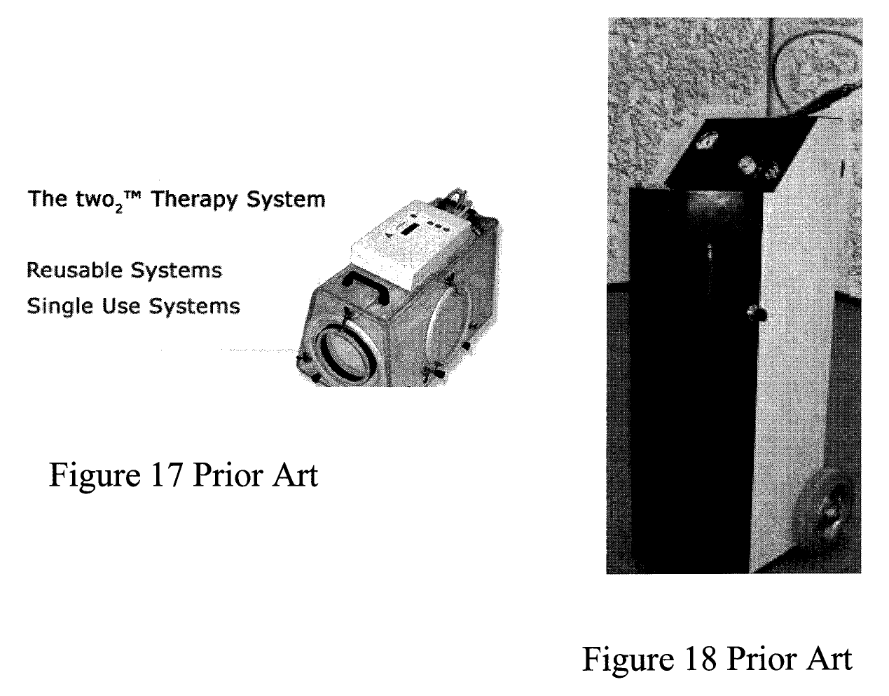

Any vacuum pumps that have been used or can be adapted for use in any NPWT, such as those illustrated in FIGS. 9 to 15, can be used in the present invention. Exemplary vacuum pumps that can be used in the present invention, include, but are not limited to, a syringe pump, a peristaltic pump, or a bellows pump. In a preferred embodiment, the vacuum pump includes a dual action pump that includes a 3-way check valve. One pump cylinder provides suction to the wound site 15 while the other cylinder removes wound effluent or exudate into the container 9. A check valve within the pump prevents negative pressure from being applied to the exudate container.

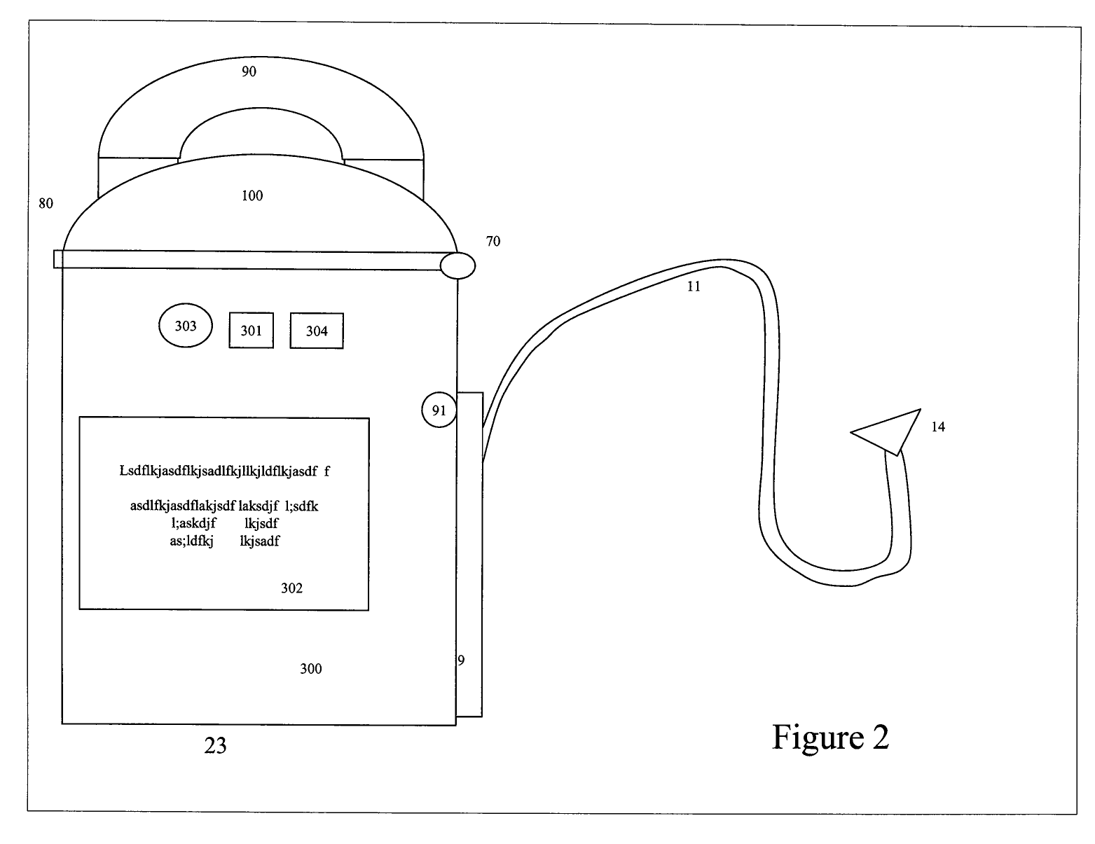

Any wound effluent containers that have been used or can be adapted for use in any NPWT can be used in the present invention. Exemplary effluent containers that can be used in the present invention, include, but are not limited to, a disposable plastic container or a reusable container. In FIG. 1, the disposable plastic container 9 is connected to the vacuum pump 7 via a connection tubing 8. A release latch (91 in FIG. 2) allows disengagement of the container 9 from the rigid housing 23 for disposal prior to re-engaging a new effluent canister. One or more sensors can be included in connection with the container to monitor the spilling or overflow of liquids from the container. A control valve can be connected with the sensors to terminate the vacuum pump 7 in case of spilling or overflow of liquids from the container.

In FIG. 1, a one-way valve 10 is included in the connective tubing 8 so as to prevent the positively pressured gaseous wound healing agent from entering the effluent container 9 and the vacuum pump 7 with subsequent loss of the positive pressure and the gaseous wound healing agent at the wound site 15. The negative pressure tubing 8 continues after the one-way valve to connect to the T-connector 6. Any flexible tubing that have been used or can be adapted for use in any NPWT can be used in the present invention.

In view of the present disclosure, it is readily apparent to a person skilled in the art that some or all components utilized in any NPWT can be modified and used in devices according to embodiments of the present invention. For example, components of a V.A.C.® system from KCI can be modified and used in the present invention according to the present disclosure.

The V.A.C.® system utilizes a sealed polyurethane foam dressing as the fluid impermeable cover. The sealed polyurethane foam dressing is attached by a tube to a vacuum pump to deliver sub-atmospheric pressure to the wound site. The treatment interface or the wound filler for a V.A.C.® system is an open-reticulated sponge that is cut to pack the size and depth of the open wound. The V.A.C.® system uses a denser sponge as the wound filler for sinus tracts and painful wounds. After the wound is filled with a sponge, the area may be covered with a semi occlusive clear drape and connected via a tube to a canister that is attached to a computer-controlled unit that applies the programmed suction. The negative pressure system pulls out stagnant fluids, such as wound exudate and collects it in a sealed canister. The sponge is said to apply macrostrain and microstrain effects to the wound bed and to induce undulations and subsequent stretching of cells which induces mitosis. The robust granulation noted within wounds treated with the V.A.C.® system is said to be due to the cellular strain and increased mitosis. The typical pressure utilized with this system is 125 mmg Hg of negative pressure.

KCI has two products released recently to the market, the V.A.C.® Activac® System and the V.A.C.® Infovac® System. The former is the acute care model and the latter the outpatient model. These two products are the third generation release. The first generation release was the original V.A.C.® Classic® and Mini-V.A.C.® and the second generation were the V.A.C.® Freedom® System and the V.A.C.® ATS® System. KCI advertises the V.A.C.® Activac® System as a portable system for advanced wound healing. The V.A.C.® Activac® System is advertised as a lightweight and portable system, designed to help patients return to work and daily activities. It has an adjustable rate of dressing-draw-down intensity for increased patient comfort, and potentially reduces the number of dressing changes and nursing visits over traditional wound care. The system includes a large 300 ml canister to minimize canister changes, and the canister is said to be easily removed and replaced. A filter system is present to minimize wound odor. The system is advertised as having long battery life (e.g., up to 12 hours), which enables patients to be mobile for a full day. The On-Screen User Guide of the system saves time, the T.R.A.C.® Pad® of the system simplifies dressing changes, and Smart Alarms™ (e.g., including audible and visible alarms) of the system help ensure patient safety. This system is, according to KCI, a lightweight and portable system that helps patients return to work and daily activities. A carrying case allows discreet delivery of therapy. KCI also has the V.A.C.® Infovac® System, which is advertised as being designed for higher acuity wounds for patients in acute care and long-term care facilities. The V.A.C.® Infovac® System features patented Therapeutic Regulated Accurate Care (T.R.A.C.®) technology for safe, controlled wound healing. This system includes audible and visual alarms, and has a battery life of approximately 4 hours. Canister volumes of 500 and 1,000 mL are available. The V.A.C.® Infovac® unit is 14.6″ (37 cm) wide by 11″ (28 cm) high by 7.1″ (18 cm) deep. It weighs 12.3 lbs. (5.6 kg). KCI also provides replacement canisters for all of the V.A.C.® systems.

In an embodiment of the present invention, components of the V.A.C.® Activac® System or the V.A.C.® Infovac® System are modified and used in the present invention in combination with a gas supply system and a controller as those described herein.

Components utilized in the recently developed Chariker, Jeter, and Tintle model of NPWT can also be modified and used in the present invention according to the present disclosure. The Chariker, Jeter, and Tintle model packed the wound with moist gauze, a wound filler, evacuated under low pressure (usually 80 mmHg) by a soft silicone drain. Most of the systems allow incorporation of either a flat drain or a round channeled drain for wounds with sinus tracts. The system is sealed with a fluid impermeable cover, i.e., a membrane similar to the Moykwas, Argenta and Shelton-Brown model. This model has been the basis for a new group of devices that have recently begun to compete with the KCI platform.

Blue Sky Medical is another company that is active in vacuum or negative pressure devices for open wounds. Blue Sky Medical markets the Versatile 1™ wound vacuum system. This system includes the Versatile 1™ Pump with a 15 foot power cord, a small 250 mL autoclavable canister, a large (800 cc) disposable canister (which uses a hoop adaptor), and a pump-canister connector. This system includes bacteria/overflow filters. This device is now licensed to Smith and Nephew as the EZCARE™ (outpatient model) and VISTA™ (acute care model). Other companies with devices similar to the Blue Sky model now exist. These competitor companies have thus far marketed their devices to utilize a simple gauze interface rather than foam. Some examples of these are the Exsudex™ Wound Drainage System by Synergy™ Healthcare, the Invia® Healing System and outpatient Liberty™ from Medela Healthcare®, Venture™ by Talley Medical, and the WoundASSIST TNP™ by AnjoHuntleigh. Some of these newer systems are not currently approved in the United States by the FDA as of yet.

In another embodiment of the present invention, components of the Versatile 1™ wound vacuum system, the EZCARE™ (outpatient model), or the VISTA™ (acute care model) are modified and used in the present invention in combination with a gas supply system and a controller as those described herein.

In yet another embodiment of the present invention, components of NPWT devices from other companies are modified and used in the present invention in combination with a gas supply system and a controller as those described herein. Devices similar to the Versatile 1™ Wound Vacuum System have now been developed by other companies. These competitor companies have thus far marketed their devices to utilize a simple gauze interface rather than foam. Some examples of these are the Exsudex™ Wound Drainage System by Synergy Healthcare (Derby, United Kingdom), the Invia® Healing System and outpatient Liberty™ from Medela Healthcare (Baar, Switzerland), Venture™ by Talley Medical (Lansing, Mich., U.S.), and the WoundASSIST® TNP by AnjoHuntleigh (Roselle, Ill., U.S.). Some of these newer systems are not yet currently approved in the United States by the FDA.

Referring to FIG. 1, the device also includes a gas supply system to apply a gaseous wound healing agent to the wound. The gas supply system includes a pressure containing source of a gaseous wound healing agent, such as a pressurized gas cylinder 18, a manual combination pressure regulator valve and gauge 1, an electronic control valve 4, a connecting tubing 3 (also called positive pressure tubing), and sensors 2 and 5.

Any pressure containing source of a gaseous wound healing agent that have been used or can be adapted for use in any topical medical gas wound treatment can be used in the present invention. For example, a pressurized metal gas cylinder 18 can be used as the source of gaseous wound healing agent in an embodiment of the present invention. The gas cylinder 18 can contain a therapeutically effective amount of a gaseous wound healing agent in a carrier gas loaded within a cradle. The gas cylinder 18 is preferably included within the rigid housing 23.

Any medical gas that is effective for topical wound treatment can be used in the present invention. Exemplary gaseous wound healing agents include, but are not limited to, carbon dioxide (CO₂), carbon monoxide (CO), nitrous oxide (N₂O), oxygen (O₂), nitric oxide (NO), H₂S, ozone (O₃), and a combination thereof.

The term “therapeutically effective amount” as used herein means that amount of a gaseous wound healing agent that accelerates or improves the healing of a wound in a subject as compared to an otherwise identical treatment without the therapeutically effective amount of the gaseous wound healing agent.

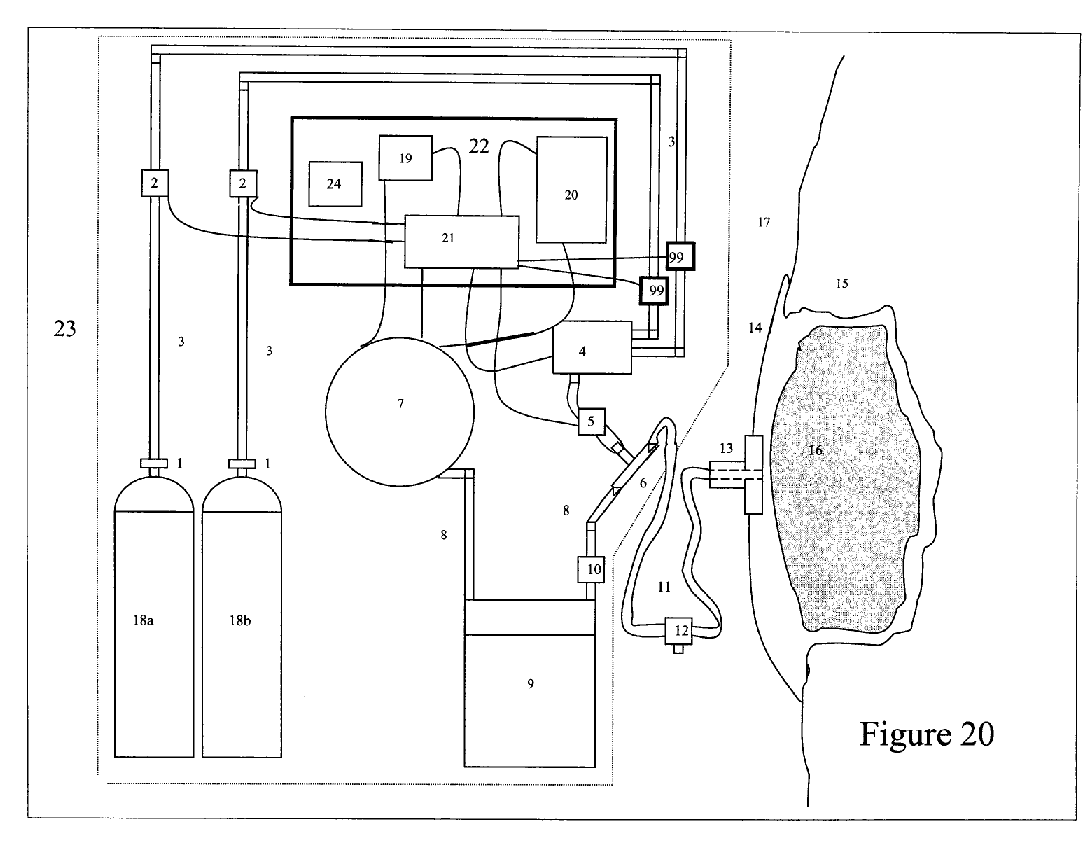

Shown in FIG. 20, in accordance with an embodiment of the present invention, the device can include multiple cylinders, such as 18a and 18b, of either different therapeutic gases, or different concentrations of the same gas, or a therapeutic gas and a carrier gas, for mixing purposes. In this embodiment, electronically controlled valves 99 would allow individual control of each gas volume and delivery of the gases either separately or simultaneously.

Each of the gas cylinders 18a and 18b is connected to a flexible tubing 3, also named positive pressure tubing, with a manual combination pressure regulator valve and gauge 1. The combination pressure regulator valve and gauge 1 reduces the cylinder pressure down to a working pressure for use with the present system and keeps track of the pressure within the gas cylinder 18a or 18b.

Referring to FIG. 1, the positive pressure tubing 3 continues on to a control valve 4. The control valve 4 controls the flow of gas from the gas cylinder 18 to the wound site 15. In an embodiment of the present invention, the control valve 4 is a solenoid valve operated by electrical signals from the controller 21. The control valve 4 can have two or more ports. In one embodiment, the control valve 4 has two ports that switch the flow of a gaseous wound healing agent on or off by electrical signals from the controller 21. The duration of time which the control valve is in the open position controls the volume of gas to the wound. The two-port valve 4 opens to allow selective insufflation of the wound site 15 with the gaseous wound healing agent, or closes to stop the gaseous wound healing agent from ingress to the wound site 15. When the control valve 4 is closed, it also acts as a check valve to prevent negative pressure from entering the positive pressure system.

In another embodiment, the control valve 4 is a three- or more than three-port valve that controls the application of more than one gaseous wound healing agents to the wound site 15. In this case, the control valve 4 switches the outflow between two or more outlet ports, i.e., two or more gaseous wound healing agents, by electrical signals from the controller 21. It can also mix two or more gaseous wound healing agents or shut off all gases together.

Referring to FIG. 1, a first pressure sensor 2 is included within the positive pressure tubing 3 to monitor the gas pressure prior to the gas entering the control valve 4. A second pressure sensor 5 is included within the positive pressure tubing 3 after the gas exiting the control valve 4.

The positive pressure tubing 3 continues after the control valve 4 to connect to the T-connector 6. Any flexible tubing that have been used or can be adapted for use in any topical medical gas wound treatment can be used in the present invention.

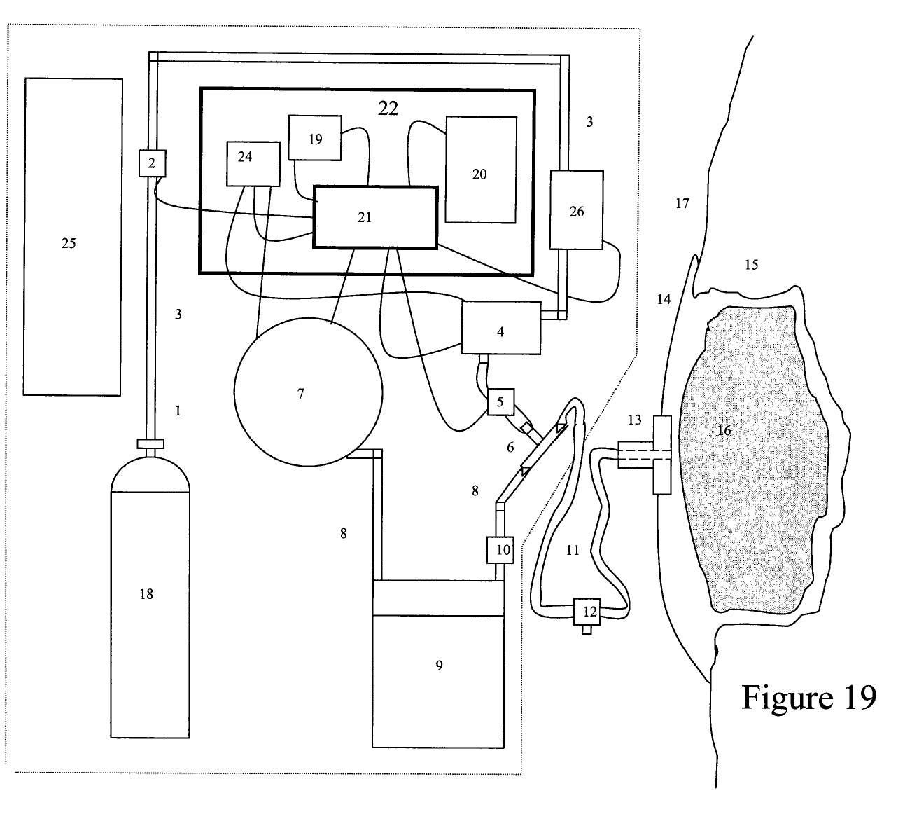

In an embodiment of the present invention that shown in FIG. 19, the pressurized gases can pass through a gas conditioning unit 26, such as a gas heating and/or humidifying unit, prior to entering the control valve 4. Any gas conditioning unit known to those skilled in the art can be used in the present invention according to the present disclosure. For example, the gas conditioning unit can be a small water-filled chamber with a heating resistance heating plate that allows pass-over humidification of the gas. The electronic controller 21 allows monitoring and control of temperature and humidity via the sensors and control valves.

In view of the present disclosure, it is readily apparent to a person skilled in the art that some or all components utilized in the topical medical gas wound treatment can be modified and used in combination with a vacuum system and a controller in a device according to embodiments of the present application present invention.

Carbon dioxide is not only able to penetrate intact skin but also the granulation tissue of the wound bed. Clinical evidence of increased granulation tissue and reduction of discharge and malodor was noted in both acute and chronic wounds from topical CO₂ wound treatment (Wollina et al, Lower Extremity Wounds (2004) 3(2): 103-106). Carboflow®, a device manufactured by Medizintechnik Karin Haaf (Gernsbach, Germany), is designed to leave the CO₂ gas coverage over the wound for 30-60 minutes for wound treatment.

In an embodiment of the present invention, components of Carboflow® are modified and utilized in the present invention in combination with a vacuum system and a controller as those described herein.

Topical oxygen has been proposed for treatment of chronic wounds, post-surgical infections, infected amputation stumps, frostbite and burns. The theory is that a lack of necessary oxygen in the injured tissues causes them not to heal. The theory continues that if adequate oxygen is supplied to the wound; it will stimulate collagen synthesis, increase fibroblast activity, increase angiogenesis, and improve leukocyte function. Examples of topical oxygen wound treatment devices include, but are not limited to, the Topical Wound Oxygen Two₂™ manufactured by AOTI (Tamarac, Fla.) and EPIFLO® from Ogenix Corp (Cleveland, Ohio, U.S.).

In another embodiment of the present invention, components of Topical Wound Oxygen Two₂™ or EPIFLO® are modified and utilized in the present invention in combination with a vacuum system and a controller as those described herein.

Topical application of gaseous nitric oxide (gNO) allows for tight control of dosing due to the short half life of NO in contact with tissues, i.e., of only 6 minutes. The gNO can be further diluted with a balance or carrier gas such as nitrogen to further lower the concentration. Modulation of the level of gNO can affect collagen and collagenase levels at the wound site. Gaseous NO at doses of less than 80 ppm produces an anti-inflammatory effect by reducing neutrophil adhesion, platelets, and pro-inflammatory cutokines in the circulating blood. Gaseous NO also acts as an endogenous antimicrobial agent. Macrophages naturally produce gNO as a host-defense mechanism against microbes, but these gNO supplies are often overcome and depleted during infection. Exogenous gaseous nitric oxide may sustain (and even enhance) the ability to defeat invading bacteria (including resistant strains) and viruses, as well as cancer cells. The combination of antimicrobial, anti-inflammatory and vasodilatory effects and the ability to manipulate the pace of collagen formation at a wound site makes gNO a very powerful tool in wound healing.

Clinical trials are currently being conducted to investigate topical gNO wound treatments. Sensormedics Corporation (Yorba Linda, Calif., U.S.), a subsidiary company of Viasys (Cardinal Health), partnering with Pulmonox Medical Inc. (Tofield, AB, Canada), is a pioneer on topical gNO wound treatment. Nitric BioTherapeutics (Bristol, Pa., U.S.) is currently running a Phase II trial involving a device to treat wounds with topical nitric oxide. Nioxx, LLC (Dickinson, Tex., U.S.) is developing topical gels that release a form of NO for wound care. ProStrakan (Galashiels, UK) is developing a topical gel to increase levels of NO for the treatment of onychomycosis.

In still another embodiment of the present invention, some components of the devices utilized for wound treatment using gNO are modified and utilized in the present invention in combination with a vacuum system and a controller as those described herein.

Referring to FIG. 1, the device further includes a programmable controller 21 that allows control of the applications of the negative pressure and the gaseous wound healing agent to the wound site 15. For example, the controller 21 allows the application of a constant negative pressure to the wound site 15 via egress of fluids and gases to the vacuum pump 7 and the application of a constant positive pressure via ingress of gas from the gas cylinder 18 via the control valve 4, any combination of intermittent negative and positive pressures, or any cyclic pattern of different gases to the wound. The controller 21 allows the user to turn the device on and off. It also allows regulation of negative and positive pressure levels at the wound site within the fluid impermeable cover 14.

In accordance with another embodiment of the present invention, the controller 21 provides intermittent negative pressure wound therapy at varying levels of suction rather than ambient pressure during the “down” cycle of negative pressure therapy.

Exemplary controllers that can be used in the present invention, include, but are not limited to electronic controller, an electric switch, a timer, a microprocessor or a combination thereof.

In an embodiment of the present invention, the controller 21 is an electronic controller. The electronic controller 21 is connected to the vacuum pump 7. It is connected to a vacuum sensor 20, which monitors vacuum levels generated by the vacuum pump 7 and relays the information to the controller 21. The signals detected by the sensor can be used to regulate the application of the negative pressure to the wound by direct algorithm preset within the controller. The electronic controller 21 is connected to a vacuum pump control circuit 19, which controls the operation of the vacuum pump 7 based on electronic signals received from the controller 21. The electronic signals for controlling the drive circuit 19 can be pre-programmed or at least partially based on information received from one or more sensors.

The electronic controller 21 is also connected to the control valve 4 in the gas supply system. It is further connected to a control valve control circuit 24, which controls the operation of the control valve 4 based on electronic signals received from the controller 21. The electronic signals for controlling the control valve circuit 24 can be pre-programmed or at least partially based on information received from one or more sensors.

Additional sensors and circuits can be connected to the controller 21 to provide additional control to the vacuum system and/or the gas supply system. Each circuit is operably connected, directly or indirectly, to the component to be controlled by the circuit. Each circuit can also be operably connected, directly or indirectly, to a sensor upon signals detected from which the operation of the component is based. The circuits can be solenoid valves operated by electrical signals from the controller 21. The circuits can have two or more ports.

In one embodiment of the present invention, the controller 21 is connected to a vacuum pump speed circuit that controls the speed of the vacuum pump 7. In an embodiment of the present invention, the controller 21 is connected to a pressure sensor, which monitors the pressure levels in the gas-supply system before or after the control valve 4 and relays the information to the controller 21. The signals detected by the sensor can be used to regulate the application of the gaseous wound healing agent by direct algorithm preset within the controller.

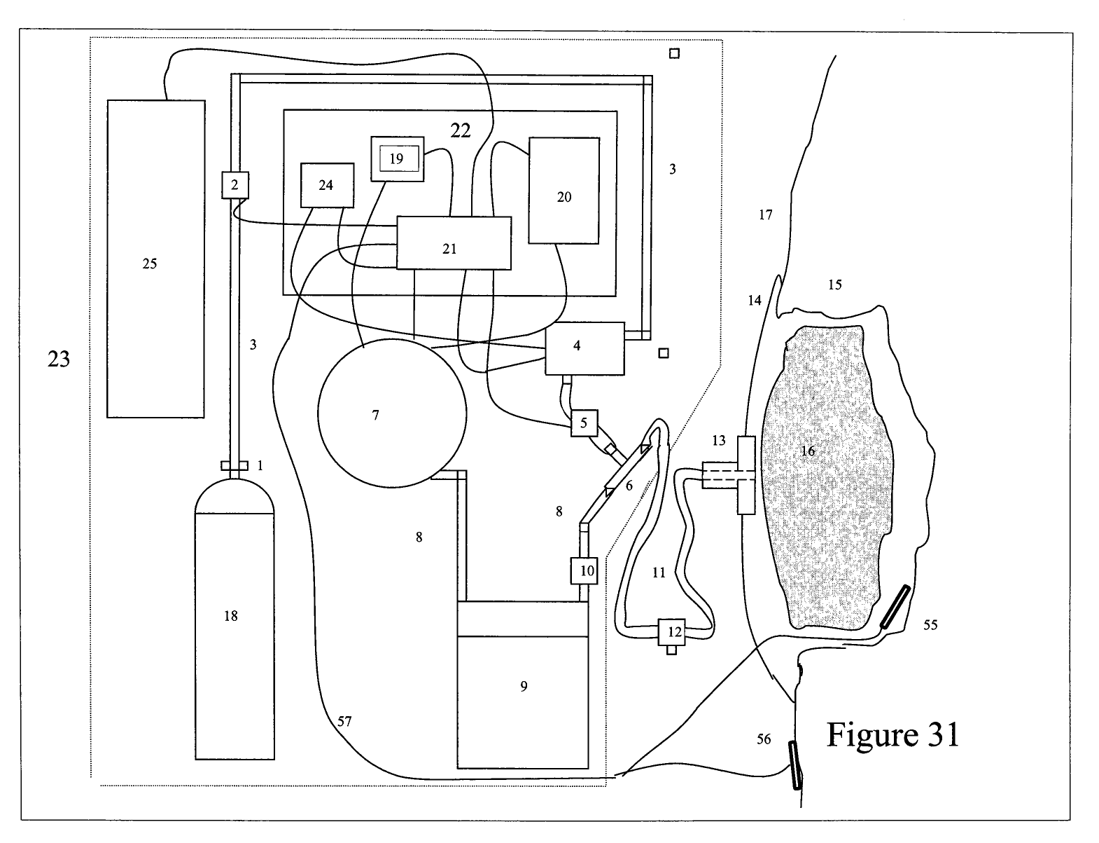

Referring to FIG. 31, in an embodiment of the present invention, the controller 21 is further connected to one or more sensors, such as 55 and/or 56, placed in or near the microenvironment created by the fluid impermeable cover 14. For example, the sensor can be placed in between of the wound and the wound filler, in between of the wound filler 16, in between of the wound filler 16 and the fluid impermeable cover 14, or on the tissue 17 surrounding the wound. The sensor detects at least one signal selected from the group consisting of pressure, pH, humidity, temperature, and the gaseous wound healing agent, and relays the detected signal to the controller 21. The sensor in the various embodiments can include, for example, carbon dioxide detectors, hygrometer, thermometer, pH level sensor, laser doppler, transcutaneous oxygen monitor, or nitric oxide display analyzer. The signals detected by the sensor can be used to optimize treatment of the wound by either manual adjustment of the various therapeutic capabilities of the device or via direct algorithm preset within the controller. The detected signal can also be shown in the instrument screen 300 connected to the controller 21 for monitoring purposes.

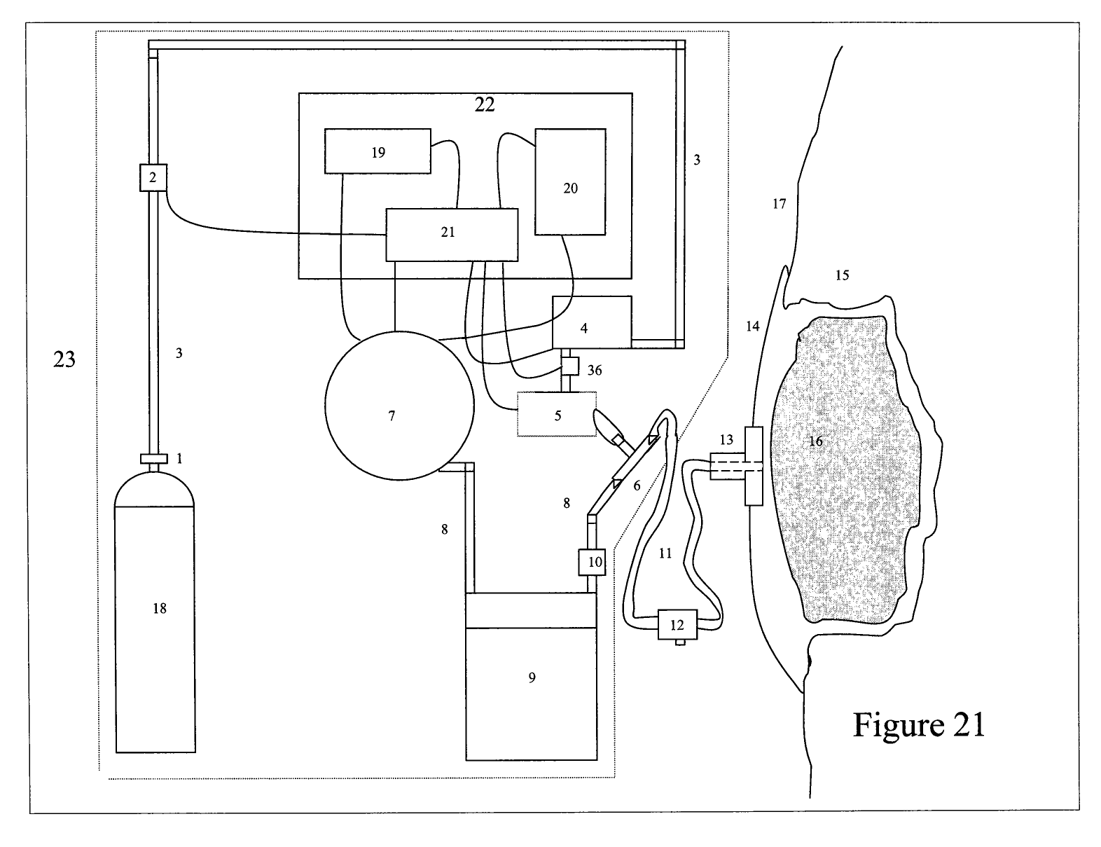

Referring to FIG. 21, in yet another embodiment of the present invention, the controller 21 is further connected with a pulsation unit 36 that allows cyclical applications of at least one of the sub-atmospheric pressure and the gaseous wound healing agent to the wound. The pulsation unit 36 is connected to at least one of the vacuum system and the gas supply system and is electronically controlled by a programmable electrical signal from the controller 21. The pulsation unit allows short cyclical application of the negative pressure or suction or short cyclical application of the gaseous wound healing agent or air to the wound site 15. This allows a low level of constant medical gas to contact the wound prior to being vacuumed out of the cover 14 with the negative pressure pump action. This also allows rapid fluctuations (pulsations) of negative and positive pressure in the microenvironment between the cover 14 and wound bed 15 and subsequent enhanced stimulation healing.

In an embodiment of the present invention, the controller 21 interfaces with the controls and liquid crystal readout on the exterior of the device. The electronic controller 21 can also provide the means to notify the user, via alarms or other warnings, of low or high gas pressures, low or high negative pressures, loss of wound filler/cover seal, low gas cylinder volume, and low battery power, etc.

The device can be powered by an alternating current, such as that of a 120 v current commonly used in the U.S. The device can also be powered by a direct current from a battery. A back-up power supply via a rechargeable battery 25 is shown in FIG. 1.

Shown in FIG. 29, in an embodiment of the present invention, the device comprises a conduit 71 that has one end placed in the microenvironment between the cover 14 and the wound 15. The one end of the conduit can be placed, for example, in between of the wound 15 and the wound filler 16, in between of the wound filler 16, or in between of the wound filler 16 and the fluid impermeable cover 14. The other end of the conduit 71 is connected with the vacuum system and/or the gas supply system via a conduit port 70 in the fluid impermeable cover 14 and the conjoined tubing 11. The conduit 71 can be a flat drain or a round channeled drain for wounds with sinus tracts. The conduit 71 can be made of soft silicone or other suitable materials. One or more openings can be included in the walls of the conduit to facilitate the communication between the surroundings of the wound and the vacuum system and/or the gas supply system.

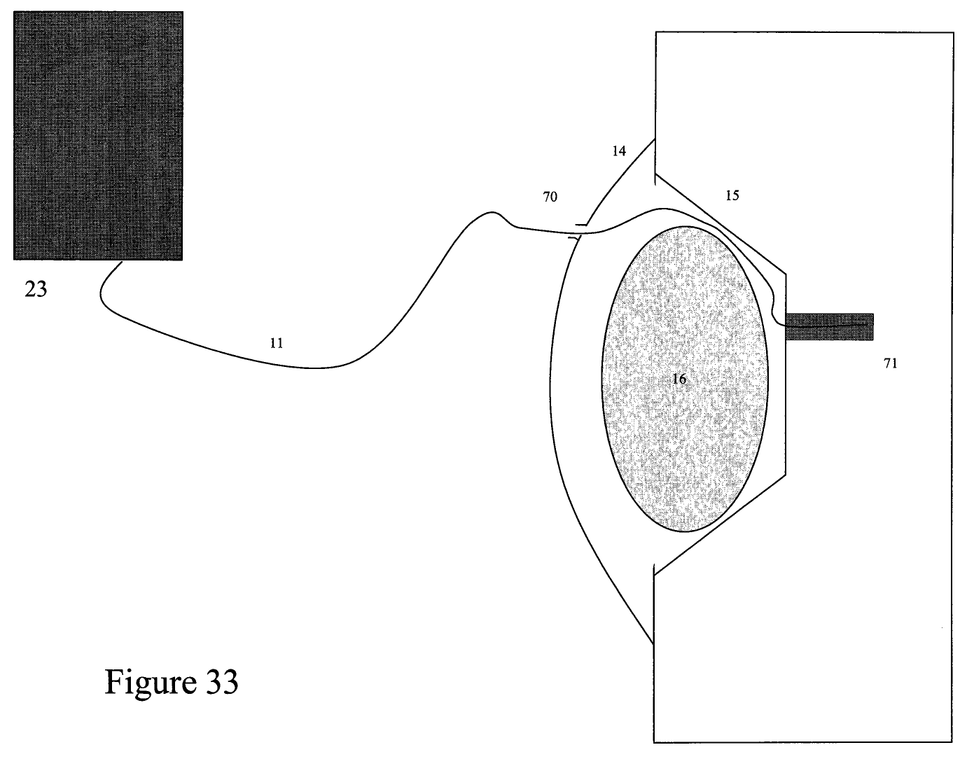

Referring to FIG. 33, in an embodiment of the present invention, the conduit 71 is used in combination with a porous pad or a moist gauze as the wound filler 16 for the treatment of an open wound that is present in conjunction with a deeper sinus tract. The fluid impermeable cover 14 allows connection of the conjoined tubing 11, via a conduit port 70, to the conduit 71, e.g., a soft drain tubing, prior to sealing over the wound filler to the peripheral intact skin.

Referring to FIGS. 24, 26 and 27, according to embodiments of the present invention, the conjoined tubing 11 from the vacuum system and the gas supply system is connected directly to the hub port or the conduit port, with subsequent positive or negative pressure therapy developing at the tip or any apertures or openings along the course of the drain tube.

Referring to FIG. 30, in another embodiment of the present invention, the conjoined tubing 11 is connected to a Y-splitter 90, which in turn connects to the hub port 13 and conduit port 70 on the fluid impermeable cover 14 via independent tubing 11a and 11b, respectively. The soft drain tubing 71 is tunneled for placement into a deep area of the wound. Positive and negative pressures can be applied at the tip or any apertures or openings along the course of the drain tubing, as well as to the wound filler material 16 via the hub port 13 independent of the soft drain tubing 71.

Referring to FIG. 29, in yet another embodiment of the present invention, the vacuum system, via tubing 8, is connected to a conduit port 70 and a soft conduit 71 to provide a negative pressure and remove exudate at the tip or any apertures or openings along the course of the drain tube 71. The gas supply system, via tubing 3, is connected to a hub port 13 on the fluid impermeable cover 14, which is independent of the conduit port 70 and the soft drain tubing 71, to apply a gaseous wound healing agent to the wound 15 through the wound filler 16.

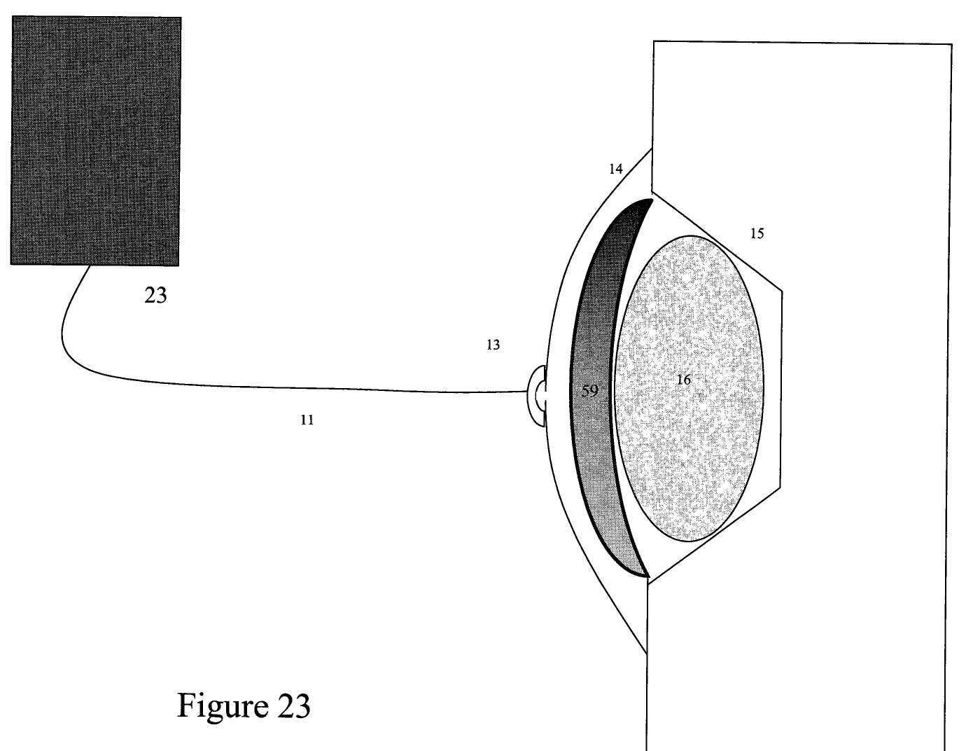

According to one embodiment of the present invention shown in FIG. 23, the device further comprises an odor filter 59 adapted to be placed in between of the wound filler 16, or in between of the wound filler 16 and the fluid impermeable cover 14. The odor filter reduces noxious odors at dressing changes. Any odor filters used in the conventional wound therapy can be used in the present invention in view of the present disclosure. For example, the filter can be made of a charcoal-activated material or other material to limit odor.

Referring to FIG. 1, the device includes a substantially rigid housing 23 that allows connection to a typical hospital bed or a portable device for the patients. According to an embodiment of the present invention, the rigid housing 23 includes most components of the device, such as the vacuum pump 7, the drainage container 9, the gas cylinder 18, the control valve 4, the controller 21, the various sensors and valves, the battery, etc.

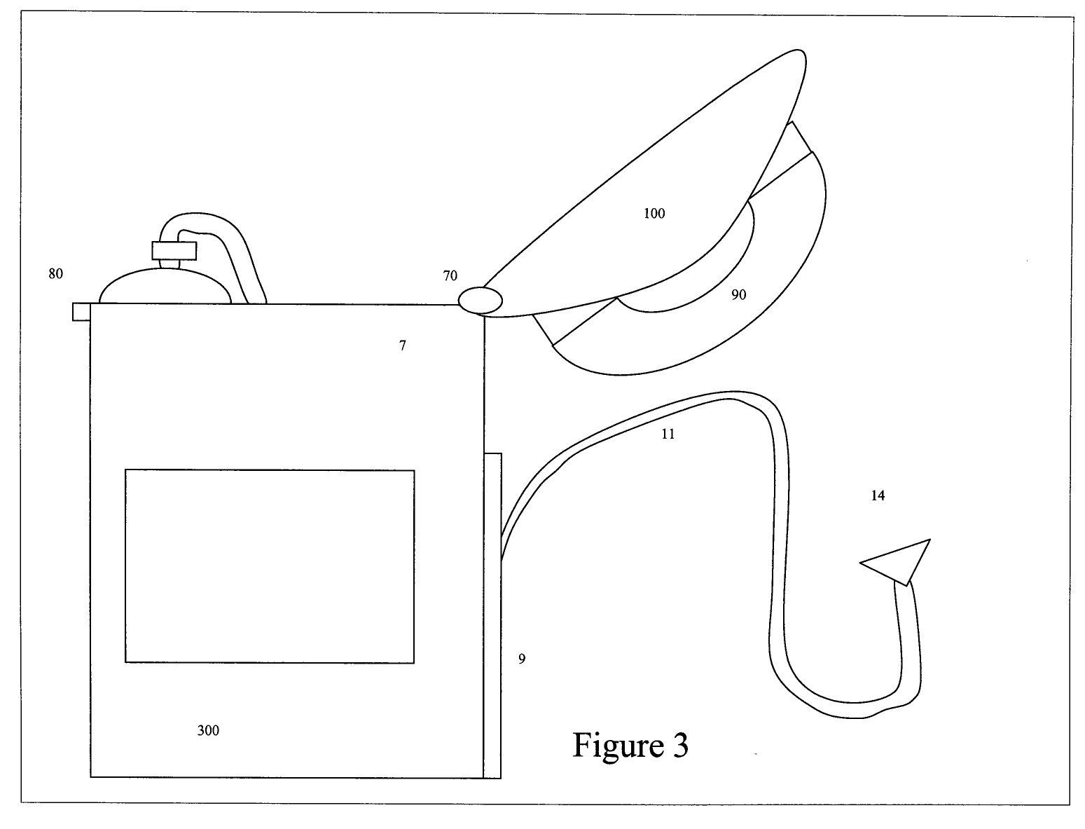

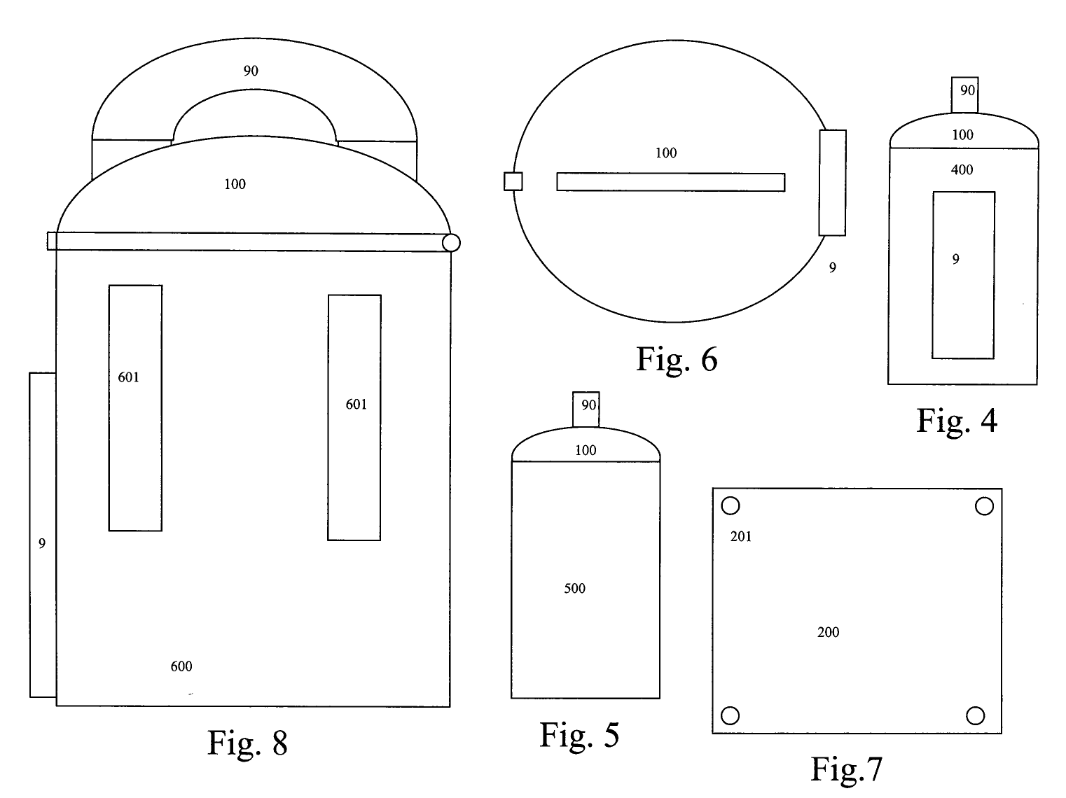

In one embodiment, the rigid housing includes a front side (FIGS. 2 and 3), a top side (FIG. 6), a bottom surface (FIG. 7), a back side (FIG. 8), a right side (FIG. 4) and a left side (FIG. 5).

Referring to FIG. 2, the front side 300 of the housing 23 can include instrumentations, such as a display screen 302, actuator buttons, e.g., 301, knobs, e.g., 303, on/off switches, e.g., 304, etc. The front side can also include a release latch 91 for the disengagement of the container 9 from the rigid housing 23. Although depicted as being included on the front surface, it should be noted that some or all of the instrumentation can also be included on other sides of the housing.

Referring to FIG. 2, the top surface of the rigid housing can include a hinge 70 and a lid 100 that can be opened and closed to allow access to the inside of the device. The hinged lid can be latched 80 to secure. It can also include a handle 90 for carrying the device.

Referring to FIG. 7, the bottom surface 200 of the rigid housing 23 can include rubber grips 201 to prevent slippage on a flat surface.

Referring to FIG. 8, the back surface 600 of the rigid housing 23 can include solid, adjustable hooks 601 for attachment to a hospital bed.

As shown in FIG. 4, the wound effluent container 9 is attached to the right side 400 of the rigid housing 23. The container 9 can be attached to the right side FIG. 4 interiorly or exteriorly. The container can also be attached to other sides of the rigid housing or being placed inside of the housing without being attached to any side of the housing. When the device is used as a hospital unit, the container 9 can also be placed separately from the rigid housing 23 to facilitate the replacement of the container or the collection of effluent from the container.

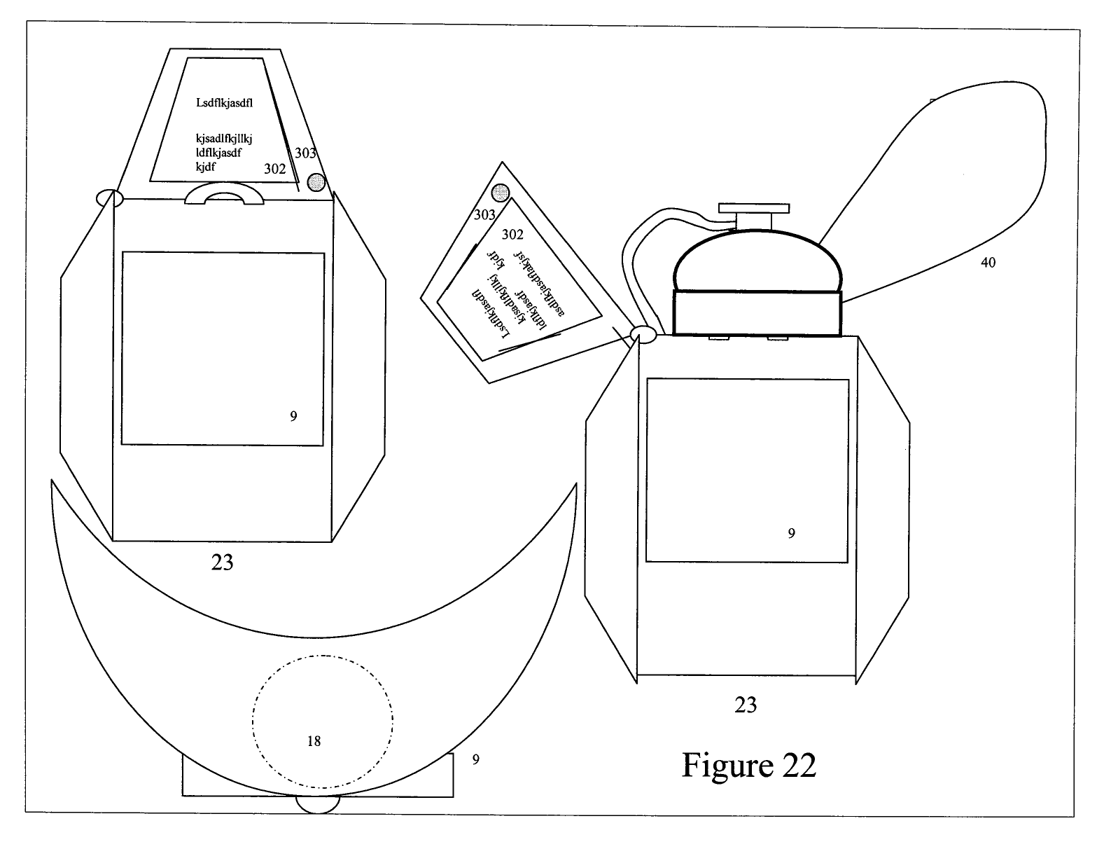

Referring to FIG. 22, according to an embodiment of the present invention, the device has a small size that allows the device to be portable with the patient in an outpatient setting. The portable and lightweight outpatient device has a smaller housing 23, smaller gas cylinder(s) 18, longer duration and high capacity battery and lower power usage of components. In this embodiment, the device weighs no more than about 3 lbs with the housing and has a dimension of no more than about 150 in 3. It should be understood that generally these dimensions and ranges are not restrictive, and a larger or smaller and heavier or lighter embodiment is possible. The battery can be rechargeable while at home or during removal and have a duration of 12-24 hours. The device housing 23 can be shaped in an ergonomic fashion such that it can be carried comfortably against the body of the user and held in place with either a belt or a shoulder strap 40 or combination of both. The device housing will allow for access to the gas cylinder 18 via a hinged top that also incorporates the instrumentations, such as a display screen 302 and control interfaces 303. A small effluent collection chamber 9 will insert into a recess in the front of the device.

Another general aspect of the invention relates to a method of promoting healing of a wound in a subject. The method comprises:
a. placing a wound filler over the wound;
b. enclosing the wound filler and the wound with a fluid impermeable cover, wherein the periphery of the fluid impermeable cover is sealed to tissue surrounding the wound;
c. applying a sub-atmospheric pressure to the wound from a vacuum system, wherein the vacuum system is operably in communication with the wound filler;
d. applying a gaseous wound healing agent to the wound from a gas supply system, wherein the gas supply system is operably in communication with the wound filler; and
e. controlling the applications of the sub-atmospheric pressure and the gaseous wound healing agent to the wound by a controller connected to the vacuum system and the gas supply system.

According to embodiments of the present invention, when positive flow of a gaseous wound healing agent enters a membrane connector on the fluid impermeable cover, the wound dressing and wound surface are bathed in warm, humid gas at low pressure with the wound filler acting as a gas diffuser. When vacuum is drawn through the membrane connector, the fluid impermeable cover collapses and uniform, moderate negative pressure is applied evenly to the wound surface. The device is designed so that it can apply continuous negative pressure therapy to the wound if necessary or clinically warranted or if there is an interruption of supply of the gaseous wound healing agent. The method is applicable to wounds, burns, infected wounds, and live tissue attachments.

By combining or coupling a negative pressure wound therapy with a topical medical gas treatment, methods according to embodiments of the present invention allow better manipulation of the process of wound healing by limiting actual exudate production, modulating collagen deposition, limiting scar formation, and increasing vasodilation. The present invention provides a powerful new treatment modality that allows clinicians to treat both chronic and acute wounds that will both allow the historic healing affects of negative pressure and true wound cascade modulation and direct action on the blood vessels surrounding the wound.

It was reported that when insufflating a cardiothoracic wound model, a gas diffuser to decrease outflow velocity of carbon dioxide provided better wound healing results than conventional tubing and that suction must not exceed gas inflow. M. Persson and J. van der Linden, Journal of Hospital Infection, (2004) 56 (2):131-136. It was also shown that insufflation with either air or CO2 decreased infection rates compared to without insufflation, that the use of a gas-diffuser lowered rates of infection as compared to an open-ended tube, that the infection rates were lowest at a location near the gas-diffuser, that higher rates of insufflation decreased infection rates, and that CO2 had lower rates of infection than air insufflation. J. van der Linden and M. Persson, J. Thorac. Cardiovasc. Surg., (2003), 125:1178-1179. In addition, it was reported that while plain gauze used as a gas diffuser may reduce outflow velocity of carbon dioxide delivered from a thin, open-ended tube, the model is not viable when the sponge is wet. J. van der Linden and M. Persson (above). This indicates that the topical gas wound treatment is influenced by various factors.

Methods according to embodiments of the present invention provides precise dosing of topical gas medication and complex cycling of intermittent variable level negative pressure, which result in improved wound healing.

In one embodiment of the present invention, at least one of the sub-atmospheric pressure and the gaseous wound healing agent is applied to the wound constantly.

In another embodiment of the present invention, at least one of the sub-atmospheric pressure and the gaseous wound healing agent is applied to the wound intermittently.

In yet another embodiment of the present invention, the sub-atmospheric pressure and the gaseous wound healing agent are applied to the wound in alternating periods for at least one operational sequence or protocol.

According to an embodiment of the present invention, at least one of the sub-atmospheric pressure and the gaseous wound healing agent is applied to the wound at a constant rate or a constant level.

According to another embodiment of the present invention, at least one of the sub-atmospheric pressure and the gaseous wound healing agent is applied to the wound at variable rates or variable levels.

According to an embodiment of the present invention, the sub-atmospheric pressure applied to the wound is about 75 mmHg below atmospheric pressure to about 125 mmHg below atmospheric pressure.

This invention will be better understood by reference to the following non-limiting example, which details an experimental model for an embodiment of the invention involving the combination of the negative pressure wound treatment and medical gas insufflations in an animal. The basic foundations of animal studies on vacuum-assisted wound treatment have been described. See, e.g., Morykwas et al., Vacuum-Assisted Closure: A New Method for Wound Control and Treatment: Animal Studies and Basic Foundation, Annals of Plastic Surgery, Vol. 38, No. 6, 1997. Those skilled in the art would readily appreciate that the protocol described below is only illustrative of the invention as described more fully in the claims which follow thereafter.

Materials and Methods

The Device

The Helios system consists of a medical-grade dressing, open-cell, polyurethane foam dressing that comes into contact with the open wound. A soft plastic tube with side ports is embedded within the foam dressing and communicates with a Y-connector. A soft plastic tube connects one arm of the Y-connector to a canister which is in turn connected to an adjustable vacuum pump. A pressure valve is embedded within the tube that closes when positive pressure is applied, thereby preventing insufflations of the canister with medical-grade gas when the pump is shut off. Another soft plastic tube connects the remaining arm of the Y-connector to small gas cylinder which is pressurized with a medical-grade gas. A pressure valve is embedded within the second soft plastic tube that closes when negative pressure is applied, thereby allowing insufflations of the foam dressing with medical gas only when the vacuum pump is not applying negative pressure to the system. The wound site and foam dressing are sealed with an adhesive drape that extends to the adjacent peri-wound skin with the tubing egressing through the drape. A pressure relief valve to prevent “over-pressurizing” of the dressing construct is embedded in the plastic tubing just prior to the foam dressing construct.

Control

The control in each experiment is an application of negative pressure wound therapy without insufflations of the foam dressing with medical grade gas. The dressing has an identical, intermittent on-off cycle of negative pressure as with the following experimental models, without insufflations during the off-cycle.

Gas Therapy

Carbon dioxide and nitric oxide gases are used in compressed gas cylinders. The experimental models allow insufflations of the dressing with the compressed gas when the negative pressure pump turns off.

Animals

Twenty (20) Charles River Crl:CD-Hr hr Hairless Rats are allowed to acclimate for 1 week prior to beginning the experimental procedures. The animals are divided into four experimental studies: (1) granulation tissue formation with control versus nitric oxide insufflations (N-5), (2) granulation tissue formation with control versus carbon dioxide insufflations (N-5), (3) flap survival with control versus nitric oxide insufflations (N-5), and (4) flap survival with control versus carbon dioxide insufflations (N-5). On the day of surgery and when needed for measurements or biopsies, the animals are sedated with an intramuscular injection of ketamine/xylazine/acepromazine (60:12-0.6 mg per kilogram). The animals are scrubbed with an antiseptic solution for surgery. Halothane (1%) is administered by inhalation for maintenance of anesthesia. All protocols and procedures are approved by the Institutional Animal and Care Use Committee, and animals are cared for under guidelines set forth in the Guidelines for Care and Use of Animals in Research.

Application of Device

The foam, tube and drape system are applied and held into place on the animal with an aquaplast saddle, Velcro straps, and a tubular, elastic bandage to prevent dislodgement of the dressing. The soft plastic tube is suspended from a pulley system above each cage that allows free-range of the animals in their cage while the dressing is in place.

Granulation Tissue Formation, Insufflations with Nitric Oxide Gas

The animals (N-5) are sedated, prepared for surgery, and two defects are created on the dorsal midline creating two circular defects 1.5 cm in diameter down to deep fascia of the muscles over the spine. The foam dressing is applied as described previously. The anterior wound dressing is connected to a combination negative and positive pressure device and the posterior wound dressing is connected to a negative pressure device only. Both devices are cycled “on” with application of negative pressure at 125 mmHg pressure for 5 minutes and “off” without negative pressure for 2 minutes. The anterior dressings receive insufflations with nitric oxide gas for the 2 minutes that the device is cycled “off”. The animals are allowed to recover from anesthesia and given food and water ad libitum.

The animals are sedated as described 48 hours after surgery, then every 48 hours thereafter, and digital wound volume measurements are obtained at each dressing change with a custom portable computer camera device with embedded laser lighting with automated imaging calibration (Aranz Medical Silhouette Mobile) until the wound base is leveled with the surrounding skin.

Granulation Tissue Formation, Insufflations with Carbon Dioxide Gas

A substantially identical granulation tissue formation experiment (N-5) is performed with carbon dioxide gas, instead of nitric oxide gas, in the above described granulation tissue formation experiment.

Adjunctive Testing, Granulation Tissue Formation Experiments

Also, at the completion of therapy, a biopsy of the wound bed is obtained from each wound in the granulation tissue formation cohort and has measurements of 4-hydroxyproline (4-Hyp) measured via high-performance liquid chromatography. 4-Hyp is a specific amino acid of collagen and widely used as a factor to estimate the collagen content in biologic specimens. High-pressure liquid chromatography (HPLC) is applied using reverse-phase elution of 7-chloro-4-nitrobenzo-2-oxa-1,3-diazole derivatives of hydroxyproline to measure collagen production by fibroblasts. Lastly, the concentration of medical gas within the foam dressing is measured utilizing specialized measuring probes (AmiNO-IV, Innovative Instruments, Inc.) embedded within the dressing and oxygen levels in the center of each wound (Oxygen Probe, Pre-Sens Precision Testing) with a needle probe.

Flap Survival, Insufflations with Nitric Oxide Gas

Four days prior to the surgery, the animals (N-5) are sedated as described. Two dorsally based 2×8 cm flaps (16 cm2) are marked on each side of the rat in indelible ink, with 4 cm between each flap. Both areas are covered by the foam dressing construct. Subatmospheric pressure of 125 mmHg is intermittently applied to the area for 5 minutes with and “off” cycle of 2 minutes until surgery four days later. One of the wounds on each rat also receives positive pressure of the area with nitric oxide gas during the “off” cycle. On the day of surgery, the rat is sedated as described and anesthesia is maintained by 1% halothane. Random-pattern flaps are created on each side of the rat following the lines previously drawn. The flaps are raised and then sutured back in place with single, interrupted 4-0 nylon sutures. The foam dressings are re-placed over the flaps and the intermittent cycle of negative pressure therapy is continued on both flaps, with positive pressurization with nitric oxide gas on only one during the “off” cycle. The animals are anesthetized as described previously 72 hours after surgery and the devices are removed. The custom portable computer camera device with embedded laser lighting with automated imaging calibration is utilized to obtain quantitative planimetry measurements of discoloration areas to allow for calculation of flap survival percentage. The devices are replaced and therapy is continued. The routine is continued at 48 hour intervals until no further necrosis or healing of necrotic areas occur. The viable surface areas of each flap are expressed as a percent of the total original flap area.

Flap Survival, Insufflations with Carbon Dioxide Gas

A substantially identical flap survival experiment (N-5) is performed with carbon dioxide gas, instead of nitric oxide gas, in the above described flap survival experiment.

Adjunctive Testing, Flap Survival Experiments

Measurements are obtained on the flap survival model animals. Laser Doppler needle probes (Moor Instruments, Devon, UK) are inserted into the subcutaneous tissue and deep back muscles adjacent to the flap areas. Blood flow measurements are recorded on a strip chart recorder Also, thermistor cutaneous temperature measurements of the flap areas is recorded including ambient room temperature prior to instituting therapy, prior to flap incision, and at each dressing change thereafter.

Statistical Analysis

A two-sided paired t-test is performed to determine significance of differences between rates of granulation tissue formation for treated versus control wounds and for percent flap survival. Mean values+/−standard deviation (SD) is presented. Statistical significance is accepted at p</=0.05. The Bonferroni correction factor for multiple testing is used.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/we claim:

1. A device for promoting healing of a wound, comprising
a. a wound filler adapted to be placed over the wound;
b. a fluid impermeable cover adapted to enclose the wound filler and the wound, wherein the periphery of the fluid impermeable cover is adapted to be sealed to tissue surrounding the wound;
c. a vacuum system adapted to apply a sub-atmospheric pressure to the wound, wherein the vacuum system is operably in communication with the wound filler;
d. a gas supply system adapted to apply a gaseous wound healing agent to the wound, wherein the gas supply system is operably in communication with the wound filler; and
e. a controller adapted for connection with the vacuum system and the gas supply system to control the application of the sub-atmospheric pressure and the application of the gaseous wound healing agent to the wound.

2. The device of claim 1, wherein the wound filler is selected from the group consisting of an open-cell foam pad, a rigid porous screen, a gauze dressing, or a polyurethane foam, a film, gels, hydrocolloids, alginates, hydrogels, polysaccharide pastes, granules, keratin proteins, and beads.

3. The device of claim 1, wherein the wound filler is predisposed with one or more agents for promotion of increased wound healing selected from the group consisting of basic fibroblast growth factor, an anti-adherence agent, a cytokine, a growth factor, and an anti-microbial.

4. The device of claim 1, wherein the fluid impermeable cover is also gas impermeable.

5. The device of claim 1, wherein the fluid impermeable cover is a transparent dressing.

6. The device of claim 1, wherein the vacuum system and the gas supply system are adapted to conjoin for connection with an opening in the fluid impermeable cover.

7. The device of claim 1, wherein the vacuum system and the gas supply system are adapted for connection with two separate openings in the fluid impermeable cover.

8. The device of claim 1, further comprising a conduit, wherein the conduit has a first end adapted to be placed in between of the wound and the wound filler, in between of the wound filler, or in between of the wound filler and the fluid impermeable cover, and the conduit has a second end adapted for connection with the vacuum source.

9. The device of claim 8, wherein the vacuum system and the gas supply system are adapted to conjoin for connection with the second end of the conduit.

10. The device of claim 8, wherein the conduit has one or more openings on the wall of the conduit.

11. The device of claim 8, wherein the conduit is selected from a soft silicone drain tube or a plastic drain tube.

12. The device of claim 1, further comprising an odor filter adapted to be placed in between of the wound filler or in between of the wound filler and the fluid impermeable cover.

13. The device of claim 1, further comprising a sensor adapted to be placed in between of the wound and the wound filler, in between of the wound filler, in between of the wound filler and the fluid impermeable cover, or on the tissue surrounding the wound, wherein the sensor is adapted to detect at least one signal selected from the group consisting of pressure, pH, humidity, temperature, and the gaseous wound healing agent, and relays the detected signal to the controller.

14. The device of claim 1, wherein the vacuum system comprises a collection device adapted to collect a fluid aspirated from the wound.

15. The device of claim 14, wherein the vacuum system comprises a pump adapted to provide the sub-atmospheric pressure to the wound and to remove the collected fluid into the collection device.

16. The device of claim 15, wherein the pump is a syringe pump, a peristaltic pump, or a bellows pump.

17. The device of claim 15, wherein the pump includes a three-way check valve.

18. The device of claim 15, wherein the vacuum system comprises a one-way valve for preventing the gaseous wound healing agent from entering the collection device and the pump.

19. The device of claim 15, wherein the pump is adapted for connection with the controller.

20. The device of claim 15, wherein the vacuum system comprises a vacuum sensor adapted to monitor the vacuum levels generated by the pump.

21. The device of claim 1, wherein the gas supply system comprises a pressure containing source of the gaseous wound healing agent.

22. The device of claim 1, wherein the gas supply system comprises at least one pressure sensor adapted to monitor the pressure of the gaseous wound healing agent.

23. The device of claim 1, wherein the gas supply system comprises a control valve adapted to control the application of the gaseous wound healing agent to the wound.

24. The device of claim 23, wherein the control valve is adapted for connection with the controller.

25. The device of claim 1, wherein the gas supply system comprises at least one of a heating unit and a humidifying unit adapted to heat and humidify the gaseous wound healing agent, respectively, prior to its application to the wound.

26. The device of claim 1, wherein the controller is operably connected with at least one control circuit that is operably connected with the component to be controlled by the controller.

27. The device of claim 26, wherein the control circuit comprises a solenoid valve.

28. The device of claim 1, wherein the controller is operably connected with at least one sensor in the vacuum system and the gas supply system.

29. The device of claim 1, wherein the controller comprises a pulsation unit adapted to allow cyclical applications of at least one of the gaseous wound healing agent and the sub-atmospheric pressure to the wound.

30. The device of claim 1, comprising a relief valve for preventing leakage due to over-vacuuming or over-pressurizing.

31. The device of claim 1, wherein the controller comprises at least one selected from the group consisting of an electrical controller, an electric switch, a timer, a microprocessor and a combination thereof.

32. The device of claim 1, wherein the controller is adapted to be programmed for at least one operational sequence or protocol.

33. The device of claim 1 being an outpatient device.

34. The device of claim 1 being an acute care device.

35. A method of promoting healing of a wound in a subject, comprising
a. placing a wound filler over the wound;
b. enclosing the wound filler and the wound with a fluid impermeable cover, wherein the periphery of the fluid impermeable cover is sealed to tissue surrounding the wound;
c. applying a sub-atmospheric pressure to the wound from a vacuum system, wherein the vacuum system is operably in communication with the wound filler;
d. applying a gaseous wound healing agent to the wound from a gas supply system, wherein the gas supply system is operably in communication with the wound filler; and
e. controlling the applications of the sub-atmospheric pressure and the gaseous wound healing agent to the wound by a controller connected to the vacuum system and the gas supply system.

36. The method of claim 35, wherein the gaseous wound healing agent is selected from the group consisting of CO₂, CO, N₂O, O₂, NO, H₂S, O₃, and a combination thereof.

37. The method of claim 35, wherein the wound is selected from a wound caused by a surgical incision, a surgical wound dehiscence, an accident, a trauma, a pathological process, and an assault.

38. The method of claim 35, wherein at least one of the sub-atmospheric pressure and the gaseous wound healing agent is applied to the wound constantly.

39. The method of claim 35, wherein at least one of the sub-atmospheric pressure and the gaseous wound healing agent is applied to the wound intermittently.

40. The method of claim 35, wherein the sub-atmospheric pressure and the gaseous wound healing agent are applied to the wound in alternating periods for at least one operational sequence or protocol.

41. The method of claim 35, wherein at least one of the sub-atmospheric pressure and the gaseous wound healing agent is applied to the wound at a constant level.

42. The method of claim 35, wherein at least one of the sub-atmospheric pressure and the gaseous wound healing agent is applied to the wound at varying levels.

43. The method of claim 35, wherein the sub-atmospheric pressure is about 75 mmHg below atmospheric pressure to about 125 mmHg below atmospheric pressure.
